(19) United States
(12) Patent Application Publication
(10) Pub. No.: US 2025/0000857 A1
(43) Pub. Date: Jan. 2, 2025

(54) SMALL MOLECULE BAX INHIBITORS AND USES THEREOF

(71) Applicants: Albert Einstein College of Medicine, Bronx, NY (US); The University of Manitoba, Winnipeg (CA)

(72) Inventors: Evripidis Gavathiotis, Roslyn, NY (US); Richard N. Kitsis, Bronx, NY (US)

(21) Appl. No.: 18/826,495

(22) Filed: Sep. 6, 2024

Related U.S. Application Data
(63) Continuation-in-part of application No. 18/595,808, filed on Mar. 5, 2024.
Application No. 18/595,808 is a continuation of application No. 16/492,300, filed on Sep. 9, 2019, now Pat. No. 11,938,128.
(60) Provisional application No. 62/469,551, filed on Mar. 10, 2017.

(51) Int. Cl. A61K 31/496; A61K 31/495; C07D 209/86; C07D 209/88; C07D 279/26; C07D 295/13

(52) U.S. Cl. CPC A61K 31/496; A61K 31/495; C07D 209/86; C07D 209/88; C07D 279/26; C07D 295/13

(57) ABSTRACT

Compounds as inhibitors of Bcl-2-associated x-protein (BAX) and pharmaceutical compositions thereof are disclosed. Also disclosed are methods of using these compounds for preserving a tissue or treating a disease or disorder in which it is desirable to inhibit BAX.

### CROSS-REFERENCE TO RELATED APPLICATIONS

This application is a continuation-in-part application of U.S. patent application Ser. No. 18/595,808 filed Mar. 5, 2024, which is a continuation of U.S. patent application Ser. No. 16/492,300 filed Sep. 9, 2019, now U.S. Pat. No. 11,938,128, which is a U.S. national entry of PCT/US2018021644 filed Mar. 9, 2018, which claims the benefit of U.S. Provisional patent application No. 62/469,551, filed on Mar. 10, 2017, the contents of each are herein incorporated by reference in their entireties.

### BACKGROUND OF THE INVENTION

Throughout this application various publications are referred to by number in parentheses. Full citations for these references may be found at the end of the specification. The disclosures of these publications, and all patents, patent application publications and books referred to herein, are hereby incorporated by reference in their entirety into the subject application to more fully describe the art to which the subject invention pertains.

Apoptosis is an evolutionarily conserved process that plays a critical role in embryonic development and tissue homeostasis. The dysregulation of apoptosis is pivotal to a number of high mortality human pathogenesis including cardiovascular diseases and neurodegenerative diseases. The pro-apoptotic Bcl-2-associated x-protein (BAX) induces mitochondrial outer-membrane permeabilization and represents a key gatekeeper and effector of mitochondrial apoptosis. In addition, BAX facilitates opening of the mitochondrial inner membrane permeability transition pore, thereby functioning as a pivotal activator of necrosis. Thus, inhibition of pro-apoptotic BAX impairs the cells' ability to initiate premature or unwanted cell death in terminally differentiated cells, including cardiomyocytes and neurons. BAX is a central mediator of both necrosis and apoptosis (17).

Myocardial infarction (MI) is a sudden event in which prolonged ischemia precipitates the deaths of myocardial cells. In ST-segment elevation MI, myocardial ischemia is precipitated by acute thrombotic occlusion of a coronary artery. In the infarct zone, necrotic deaths of cardiomyocytes and non-myocytes predominate, beginning within ˜1 h of ischemia and continuing for <1 day. In addition, a delayed wave of apoptosis takes place in the peri-infarct zone peaking at ˜24 h in myocardial infarction/reperfusion (MI/R). Both forms of cell death play important roles in the evolution of the infarct (1-3). Necrosis is responsible for the drastic decrease in cellularity within the infarct zone and for eliciting downstream tissue responses such as inflammation, matrix remodeling, and later fibrosis (4), and apoptosis in the peri-infarct zone is a major component of early post-infarct remodeling (5). The amount of cardiac damage over the first ˜24-48 h of MI, “infarct size”, is the major determinant of post-MI chronic heart failure and mortality in humans and experimental animals (6,7). As MIs are the proximate cause of ˜50% of heart failure cases, therapeutic interventions to limit cardiac damage sustained over just the first 24-48 hours present an opportunity to impact the incidence of heart failure.

Current treatments for MI include: (a) drugs that reduce myocardial oxygen demand (e.g. β-adrenergic receptor blockers) (8-10); and (b) reperfusion, usually through angioplasty/stenting. While both therapies demonstrate efficacy (11-12), considerable mortality remains. The development of effective treatments has proved challenging. Unsuccessful examples include anti-oxidants such as superoxide dismutase (13), Na⁺/H⁺ exchange inhibitors (14), and various anti-neutrophil antibodies (15,16).

The present invention address the need for inhibitors of BAX that can be used to treat MI and other indications in which inhibition of premature or unwanted cell death is desirable, such as, for example, chemotherapy-induced cardiomyopathy.

### SUMMARY OF THE INVENTION

An aspect of the patent document provides a compound of Formula A, or a pharmaceutically acceptable salt thereof,

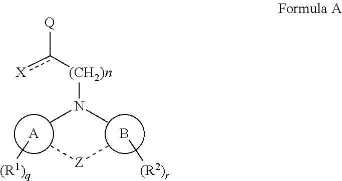

Wherein
A is optionally substituted phenyl, naphthyl, indene, or a 5-10-membered heteroaromatic ring having 1, 2 or 3 N atoms in the heteroaromatic ring;
B is optionally substituted phenyl, naphthyl, indene, or a 5-10-membered heteroaromatic ring having 1, 2 or 3 N atoms in the heteroaromatic ring; or a 5-6-membered aliphatic ring with up to 3 heteroatoms;
R¹ and R² in each instance are independently selected from the group consisting of C1-C5 alkyl, F, Cl, Br, I, CN, NO₂, N(R⁴)₂, OR⁴, CF₃, COOH, COOR⁴, NHR⁴, OCR⁴, OCOR⁴, OR⁴, SR⁴, SOR⁴, and SO₂R⁴;
X is H, NH₂, OH, O, F, Cl, Br, I, CN, SH, NO₂, N(R⁴)₂, OR, CF₃, COOH, R⁴, COOR⁴, NHR⁴, OCR⁴, OCOR⁴, OR⁴, SR⁴, SOR⁴, or SO₂R⁴, wherein the bond between X and the main scaffold is a single bond or a double bond; alternatively, X is NR⁴ and the bond between X and the main scaffold is a double bond;
Q is

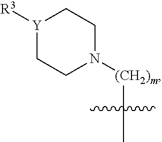

(CH₂)ₘN((CH₂)ₒR⁵)₂, COH, COOH, or CH₂NH(CH₂)₁OH;
R³ is none, H, C1-C6 alkyl, R4(C═O), or (CH₂)ₚOH;
R⁴ in each instance is independently H or C1-C3 alkyl;
R⁵ in each instance is independently OH, SH, NR4₂ or R4;
the dashed lines between A and Z and between B and Z indicates an optional bond;
Y is O, S, N or CH;
each of the dashed lines represents an optional bond in compliance with valency rule;
Z is void, O, S, NR4, CHR4, S(O)₂, C(Me)₂ or C(O), provided that when both of the dashed lines are absent, Z is void;
each l, m, n, o and p is independently 1, 2 or 3;
r is 0, 1, 2, 3 or 4; and
q is 0, 1, 2, 3 or 4.

Another aspect provides a method of treating a disease associate with mitochondrial outer-membrane permeabilization in a subject, comprising administering to the subject in need thereof a therapeutically effective amount of the compound or the pharmaceutically acceptable salt thereof disclosed herein.

Another aspect provides a method of storing a biological material ex vivo or prolonging the viability of the biological material ex vivo for a period of time, comprising contacting the biological sample during the period with an effective amount of the compound or the pharmaceutically acceptable salt thereof disclosed herein.

### BRIEF DESCRIPTION OF THE DRAWINGS

FIG. 1. Deletion of BAX in knockout mice reduces myocardial infarct size in vivo. Number of mice shown in circles. Left column in each pair represents data from wild type mice, while right column represents data from BAX knock-out mice. AAR—area at risk, INF—infarct size, LV—left ventricle.

FIG. 2. Compounds BAI-1 and BAI-2 inhibit membrane permeabilization induced by tBID-activated BAX. Maximum permeabilization of tBID-induced BAX activation was recorded at ˜60%. Maximum permeabilization of BAX alone was recorded at ˜10%. Another compound, BAI-3, was not effective to inhibit tBID-induced BAX activation. tBID, BAI-1, BAI-2, and BAI-3 alone do not stimulate fluorophore release (not shown). Structures of BAI-1 and BAI-2 are illustrated in FIG. 3A; structure of BAI-3 shown in FIG. 3C.

FIG. 3A-3C. Structures of compounds.

FIG. 4. NMR CSP-guided docking of BAI-1 with the BAX monomer structure using Glide (Shrodinger). BAI-1 molecules form a number of hydrophobic contacts, cation-x and electrostatic interactions in the proposed binding site, stabilizing interactions of the hydrophobic core of the BAX structure.

FIG. 5. Effects of BAI-1 on tBID-induced BAX oligomerization.

FIG. 6A-6C. BAI-1 potently inhibits primary (A) neonatal and (B) adult cardiomyocyte death induced by hypoxia and hypoxia/reoxygenation (H/R), respectively. Cell death was assessed using calcein AM (alive) and ethidium homodimer (dead) staining. (C) BAI1 inhibits hypoxia-induced loss of inner mitochondrial membrane potential (Δψm) in neonatal cardiomyocytes.

FIG. 7. BAI-A19 inhibition of BAX activation in vivo assayed with 6A7antibody staining.

FIG. 8. BAI compounds tested in BAX inhibition assay using tBID-induced BAX activation in liposomal membranes. Structures of the compounds are shown in FIG. 3.

FIG. 9. BAI-A19 potently inhibits primary neonatal cardiomyocyte death induced by hypoxia, whereas the inactive analog, BAI1-A20, does not. Cell death was assessed using calcein AM (alive) and ethidium homodimer (dead) staining.

FIG. 10. BAI-A22 potently inhibits primary neonatal cardiomyocyte death induced by hypoxia.

FIG. 11A-11B. BAI-A22 pharmacokinetics. BAI-A22 was injected intravenously in male Sprague DawleyR rats at 1 mg/kg dose and blood plasma and heart tissue were collected at different time points, and drug levels measured by LC-MS/MS. (A) Average BAI-A22 concentrations in blood plasma. (B) Average BAI-A22 concentrations in heart tissue.

FIG. 12. Simulated heart concentration of BAI-A22. Dose Regimen to reach 3000 μg/mL heart concentration: 650 μg of IV bolus and 132 μg/h IV infusion. Dotted lines indicate the therapeutic window.

FIG. 13. BAI-1 inhibits tBID-induced BAX mediated cytochrome c release from isolated cardiac mitochondria. The levels of cytochrome c were quantified in mitochondrial and soluble fractions after incubation of mitochondria with tBID, BAX without and with BAI-1.

FIG. 14. BAI-1 inhibits TNFa-induced BAX-mediated apoptotic cell death in mouse embryonic fibroblasts. Inhibition of BAI-1 required BAX but not BAK.

FIG. 15. BAI-1 inhibits Ionomycin-induced BAX-mediated necrotic cell death in mouse embryonic fibroblasts as measured by the loss of TMRE fluorescence using FACS analysis.

FIG. 16. BAI-1 inhibits doxorubicin-induced cell death in rat neonetal cardiomyocytes. Cell death was assessed using calcein AM (alive) and ethidium homodimer (dead) staining.

FIG. 17. BAI-1 inhibits doxorubicin-induced cell death in human cardiomyocytes derived from human induced pluripotent stem cells. Cell death was assessed using annexin V staining.

FIG. 18. BAI-A22 inhibits doxorubicin-induced apoptosis in mouse embryonic fibroblasts. Apoptosis was induced with either staurosporine (STS) or pro-apoptotic ABT-737 drug.

FIG. 19A-19C. BAX inhibitor effect on heart function in doxorubicin-treated mice. (A) Effects of doxorubicin with or without BAX inhibitors BAI-1, BAI-A2, and BAI-A22 on ejection fraction in acute doxorubicin model. Groups were compared using one-way ANOVA followed by Tukey's multiple comparisons test. (B) Effect of doxorubicin with or without BAX inhibitor BAI-1 on ejection fraction in acute doxorubicin model. Groups were compared using one-way ANOVA followed Dunnett's multiple comparisons test. (C) Effect of doxorubicin with or without BAX inhibitor BAI-A22 on ejection fraction in acute doxorubicin model. Groups were compared using one-way ANOVA followed Dunnett's multiple comparisons test.

FIG. 20A-20B. BAI-1 inhibits cardiomyocyte apoptosis and necrotic cell death. (A) TUNEL assay was used to measure apoptosis in neonatal rat cardiomyocytes (NRCM) under normoxia and hypoxia with or without 1 μM BAI-1. Quantification of percentage of TUNEL positive cells. (B) NRCM were pre-treated with varying concentrations of BAI-1 and cultured under normoxic or hypoxic conditions. BAI-1 dose response. Percentage of dead cells represents percentage of ethidium homodimer-1 (EthD) positive cells.

FIG. 21A-21C. BAI-A1, BAI-A2 and BAI-A21 inhibit cardiomyocyte necrotic cell death. NRCM were pre-treated with varying concentrations of BAI-A1 (A), BAI-A2 (B) or BAI-A21 (C) and cultured under normoxic or hypoxic conditions. Percentage of dead cells represents percentage of EthD positive cells.

FIG. 22A-22B. BAI-1 and BAI-A1 inhibit adult cardiomyocyte necrosis. Adult rat cardiomyocytes were pre-treated with varying concentrations of BAI-1 (A) or BAI-A1 (B) and subjected to hypoxia and reoxygenation (H/R). Percentage of dead cells represents percentage of EthD positive cells.

FIG. 23. BAI-A2 inhibits doxorubicin-induced cardiomyocyte necrosis. NRCM were pre-treated with varying concentrations of A2 and then stimulated with 10 μM doxorubicin for 18 hr. A2 dose response. Percentage of dead cells represents percentage of EthD positive cells.

FIG. 24A-24C. BAI-1 reduces doxorubicin-induced cardiac fibrosis, apoptosis and necrosis. (A) Hearts were collected from mice in the acute doxorubicin model and sectioned to measure fibrosis using Masson Trichrome staining. Percentage of area with blue collagenous stain per field. (B) Hearts were collected from mice in the acute doxorubicin model and sectioned to stain for apoptosis marker, TUNEL. Percentage of TUNEL positive nuclei per field. (C) Hearts were collected from mice in the acute doxorubicin model and sectioned to immunostain for high motility group box 1 (HMGB1), loss of which indicates necrosis. Percentage of nuclei that lost HMGB1 per field.

FIG. 25A-25B. BAI-1 effects on doxorubicin-induced cardiac apoptosis in chronic model. (A) Hearts were collected from mice in the chronic doxorubicin model and sectioned to stain for the apoptosis marker, TUNEL. Percentage of TUNEL positive nuclei per field. (B) Hearts were collected from mice in the chronic doxorubicin model and sectioned to immunostain for HMGB1, loss of which indicates necrosis. Percentage of nuclei that lost HMGB1 per field.

FIG. 26. Chronic model of doxorubicin and trastuzumab combination therapy. Experimental scheme for chronic model of doxorubicin and trastuzumab-induced cardiomyopathy. In doxorubicin group, mice were administered 3 mg/kg doxorubicin for 8 injections. In doxorubicin plus trastuzumab group, mice were administered 1 week later with 2 mg/kg trastuzumab for 6 injections. Echocardiography was performed at week 8 from the start of experiment. Trastuzumab (TRZ).

FIG. 27. BAI-1 protects the heart against doxorubicin and trastuzumab combination therapy. Echocardiographic assessment of cardiac function and dimensions showing significant rescue by BAI-1. Ejection fraction (EF); fractional shortening (FS); stroke volume (SV). Trastuzumab (TRZ). Saline, n=6; DOX, n=8; DOX+TRZ, n=8; DOX+TRZ+BAI1, n=8 mice.

FIG. 28. BAI-1 does not inhibit doxorubicin-induced breast cancer cell death. Human breast cancer cell lines LM2, MDA-MB-231, 3475 and MCF-7 were treated with doxorubicin without or with varying concentrations of BAI-1. Percentage of cell viability is relative to 100% viability of an untreated control group (not shown). BAI-1 did not interfere with doxorubicin-induced killing of any of the breast cancer cells. Cell viability was assessed using CytoTox-Glo Cytotoxicity Assay (Promega).

FIG. 29. Effects of BAI-1 on doxorubicin-induced AML cell viability in vitro. Human AML cell lines THP-1, HL-60 and MOLM-13 were treated with doxorubicin without or with varying concentrations of BAI-1. Percentage of cell viability is relative to 100% viability of an untreated control group (not shown). BAI-1 did not interfere with doxorubicin-induced killing of any of the AML cells.

FIG. 30 shows the synthesis of an example compound.

FIG. 31 shows the synthesis of an example compound.

FIG. 32 shows the synthesis of an intermediate compound.

### DETAILED DESCRIPTION OF THE INVENTION

The invention provides compounds as inhibitors of Bcl-2-associated x-protein (BAX). Also provided are methods of treating a disease or condition in a subject in which it is desirable to inhibit BAX by administering to the subject one or more of the BAX inhibitors disclosed herein.

An aspect of the patent document provides a compound of Formula A, or a pharmaceutically acceptable salt thereof,

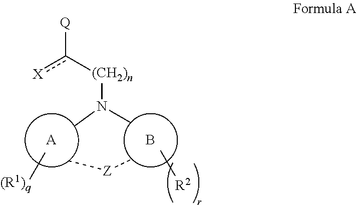

Wherein
A is phenyl, naphthyl, indene, or a 5-10-membered heteroaromatic ring having 1, 2 or 3 N atoms in the heteroaromatic ring;
B is phenyl, naphthyl, indene, or a 5-10-membered heteroaromatic ring having 1, 2 or 3 N atoms in the heteroaromatic ring; or a 5-6-membered aliphatic ring with up to 3 heteroatoms;
A and B are independently and optionally substituted with one or more of C1-C5 alkyl, halo C1-C5 alkyl (e.g. CF2, CF3, CH2CH2F), F, Cl, Br, I, CN, N (R⁴)₂, OR⁴, CF₃, COOH, and COOR⁴, NH₂, OH, F, CI, Br, I, CN, SH, NO₂, N(R⁴)₂, CF₃, COOH, R⁴, COOR⁴, NHR⁴, OCR⁴, OCOR⁴, OR⁴, SR⁴, SOR⁴, and SO₂R⁴;
R¹ and R² in each instance are independently selected from the group consisting of C1-C5 alkyl, F, Cl, Br, I, CN, NO₂, N(R⁴)₂, OR⁴, CF₃, COOH, COOR⁴, NHR⁴, OCR⁴, OCOR⁴, OR⁴, SR⁴, SOR⁴, and SO₂R⁴;
X is H, NH₂, OH, O, F, Cl, Br, I, CN, SH, NO₂, N(R⁴)₂, OR, CF₃, COOH, R⁴, COOR⁴, NHR⁴, OCR⁴, OCOR⁴, OR⁴, SR⁴, SOR⁴, or SO₂R⁴, wherein the bond between X and the main scaffold is a single bond or a double bond; alternatively, X is NR⁴ and the bond between X and the main scaffold is a double bond;
Q is

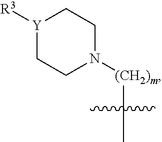

(CH₂)ₘN((CH₂)ₒR⁵)₂, COH, COOH, or CH₂NH(CH₂)₁OH;
R³ is none, H, C1-C6 alkyl, R4(C═O), or (CH₂)ₚOH;
R⁴ in each instance is independently H or C1-C3 alkyl;
R⁵ in each instance is independently OH, SH, NR4₂ or R4;
the dashed lines between A and Z and between B and Z indicates an optional bond;
Y is O, S, N or CH;
each of the dashed lines represents an optional bond in compliance with valency rule;
Z is void, O, S, NR4, CHR4, S(O)₂, C(Me)₂ or C(O), provided that when both of the dashed lines are absent, Z is void;
each 1, m, n, o and p is independently 1, 2 or 3;
r is 0, 1, 2, 3 or 4; and
q is 0, 1, 2, 3 or 4.

In some embodiments, Z is present and each of the dashed lines represents a bond. In some embodiments, Z is void and the dashed lines together represent a bond between ring A and ring B. In some embodiments, Z is void and the dashed lines are absent.

In some embodiments, A and B are different rings (not considering their respective substituents). For example, one of them is an optionally substituted phenyl, while the other is an optionally substituted naphthyl, indene, or a 5-10-membered heteroaromatic ring (e.g. indole, quinoline, quinazoline, etc.).

In some embodiments, R¹ and R² in each instance are independently selected from the group consisting of C1-C5 alkyl, F, Cl, Br and CF₃.

In some embodiments, r is 0 or 1, q is 0 or 1.

In some embodiments, X is OH, A is naphthyl, indene, or 10-membered heteroaromatic ring having 1, 2 or 3 N atoms in the heteroaromatic ring, wherein the naphthyl, indene, or 10-membered heteroaromatic ring is optionally substituted with one or more substituents selected from the group consisting of C1-C5 alkyl, halo C1-C5 alkyl (e.gt CF2, CF3, CH2CH2F), F, Cl, Br, I, CN, N(R⁴)₂, OR⁴, CF₃, COOH, and COOR⁴. In some embodiments, A is optionally substituted naphthyl or indene. In some embodiments, B is an optionally substituted phenyl.

The nitrogen (not the optional Z) between A and B can be bonded to any atom on ring A and ring B as long as the bonding complies with valency rules. Nonlimiting examples of rings for A and/or B include the following, each of which can be further optionally substituted.

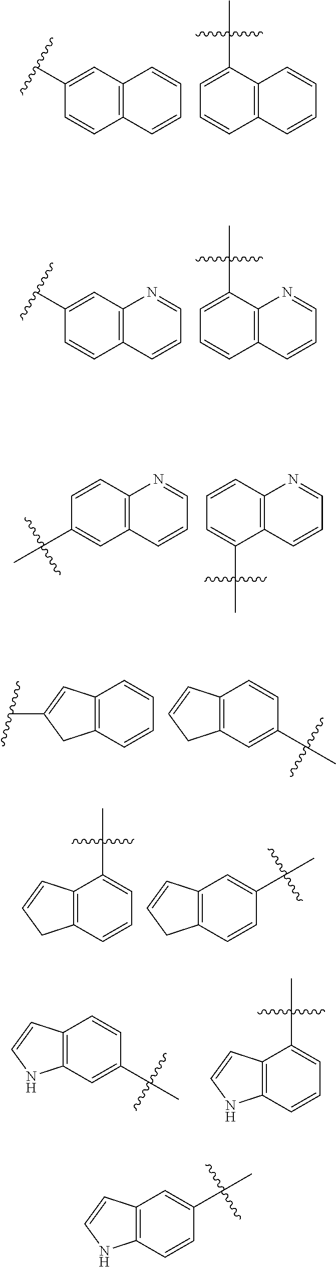

Additional examples of rings for A and/or B include the following, each of which can be further optionally substituted.

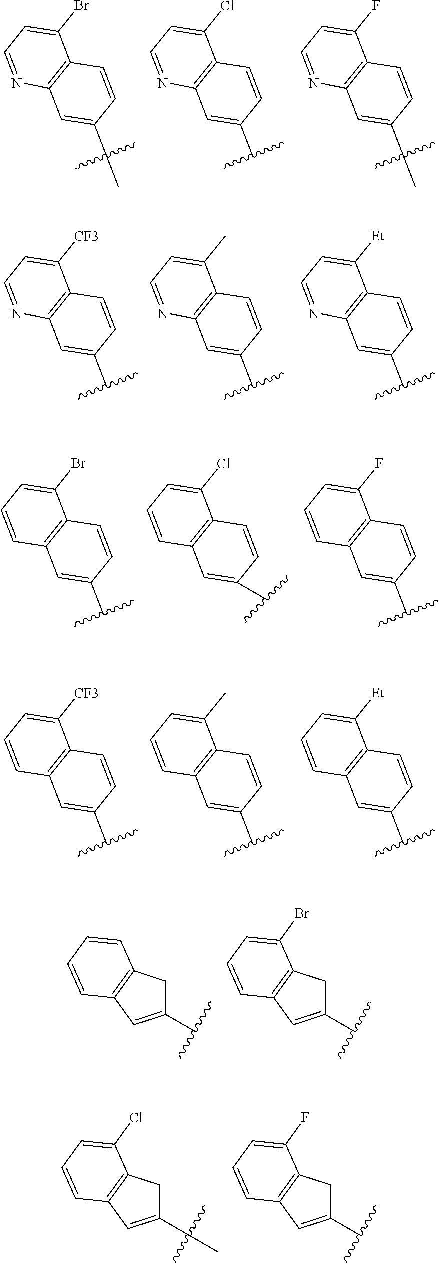

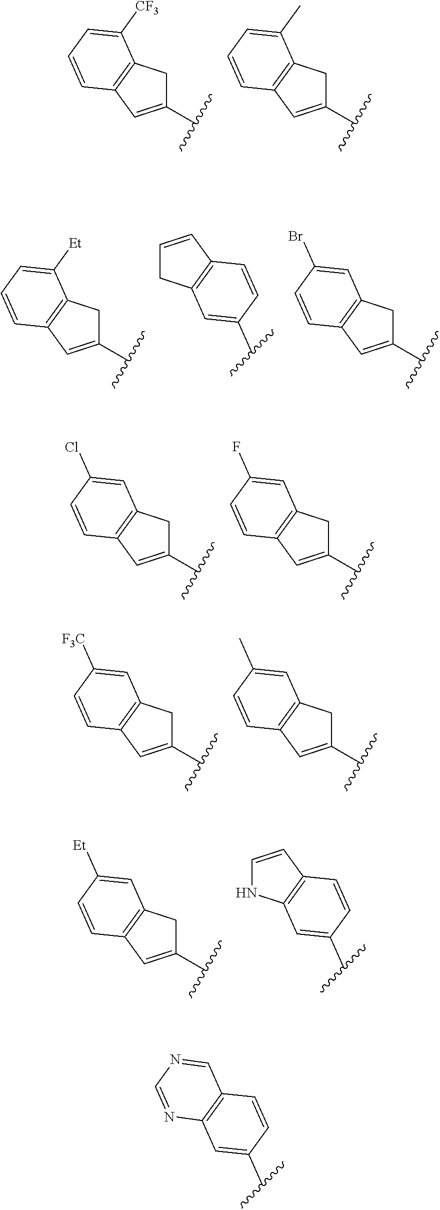

In some embodiments, the compound is represented by Formula A-1, wherein ring A and ring B are independently selected from the groups described above.

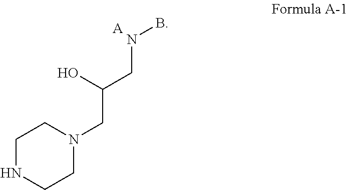

In some embodiments of A-1, Ring A is selected from the groups described above, and Ring B is 4-bromo-phenyl.

In some embodiments, A is

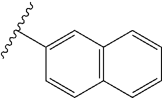 [SMILES: *c1ccc2ccccc2c1]

which can be optionally substituted

In some embodiments, A is optionally substituted 10-membered heteroaromatic ring having 1 or 2 N atoms. In some embodiments, A is optionally substituted indole, quinoline or quinazoline. In some embodiments, A is

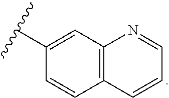 [SMILES: *c1ccc2cccnc2c1]

In some embodiments, the compound is represented by Formula A-2.

Nonlimiting examples of Formula A-2 include the following.

A-2

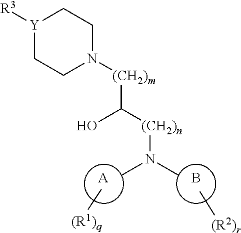

| | | m | n | 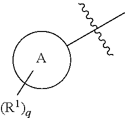 | 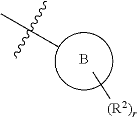 |
|---|---|---|---|---|---|
| 1 | NMe | 1 | 1 | 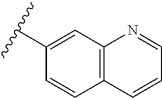 [SMILES: *c1ccc2cccnc2c1] | 4-Br-phenyl |
| 2 | NH | 1 | 2 | 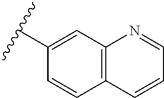 [SMILES: *c1ccc2cccnc2c1] | 4-Cl-phenyl |
| 3 | NH | 2 | 1 | 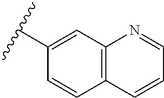 [SMILES: *c1ccc2cccnc2c1] | 4-CF₃-phenyl |
| 4 | NH | 1 | 2 | 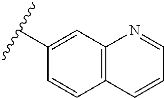 [SMILES: *c1ccc2cccnc2c1] | 4-Et-phenyl |
| 5 | NH | 1 | 2 | 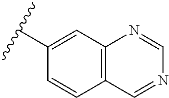 [SMILES: *c1ccc2cncnc2c1] | 2-F-4-Br-phenyl |
| 6 | NH | 1 | 1 | 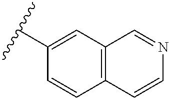 [SMILES: *c1ccc2ccncc2c1] | 4-Br-phenyl |
| 7 | NH | 2 | 1 | 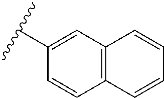 [SMILES: *c1ccc2ccccc2c1] | 4-Br-phenyl |
| 8 | NH | 1 | 2 | 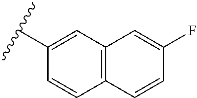 [SMILES: *c1ccc2ccc(F)cc2c1] | 4-Cl-phenyl |
| 9 | NH | 1 | 2 | 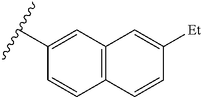 [SMILES: *c1ccc2ccc(CC)cc2c1] | 4-CF₃-phenyl |
| 10 | NH | 1 | 1 | 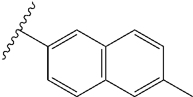 [SMILES: *c1ccc2cc(C)ccc2c1] | 4-Br-phenyl |
| 11 | NH | 1 | 1 | 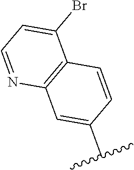 [SMILES: *c1ccc2c(Br)ccnc2c1] | 4-Br-phenyl |
| 12 | NMe | 1 | 1 | 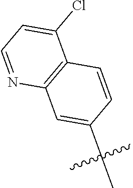 [SMILES: *c1ccc2c(Cl)ccnc2c1] | 4-Cl-phenyl |
| 13 | NMe | 1 | 1 | 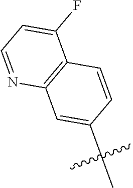 [SMILES: *c1ccc2c(F)ccnc2c1] | 4-CF₃-phenyl |
| 14 | NH | 1 | 2 | 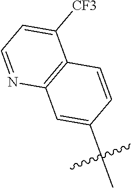 [SMILES: *c1ccc2c(C(F)(F)F)ccnc2c1] | 4-Et-phenyl |
| 15 | NH | 2 | 1 | 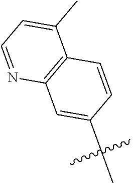 [SMILES: *c1ccc2c(C)ccnc2c1] | 2-F-4-Br-phenyl |
| 16 | NH | 1 | 2 | 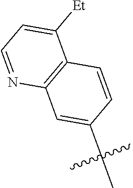 [SMILES: *c1ccc2c(CC)ccnc2c1] | 4-Br-phenyl |
| 17 | NH | 1 | 2 | 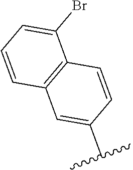 [SMILES: *c1ccc2c(Br)cccc2c1] | 4-Br-phenyl |
| 18 | NH | 1 | 1 | 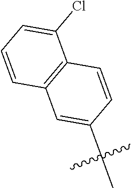 [SMILES: *c1ccc2c(Cl)cccc2c1] | 4-Br-phenyl |
| 19 | NH | 1 | 1 | 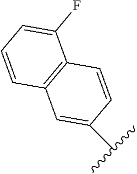 [SMILES: *c1ccc2c(F)cccc2c1] | 4-Cl-phenyl |
| 20 | NH | 2 | 1 | 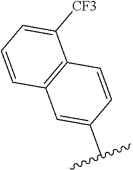 [SMILES: *c1ccc2c(C(F)(F)F)cccc2c1] | 4-CF₃-phenyl |
| 21 | NH | 1 | 2 | 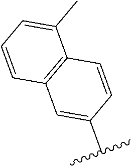 [SMILES: *c1ccc2c(C)cccc2c1] | 4-Br-phenyl |
| 22 | NMe | 1 | 1 | 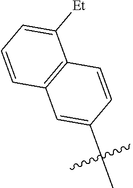 [SMILES: *c1ccc2c(CC)cccc2c1] | 4-Br-phenyl |
| 23 | NH | 1 | 2 | 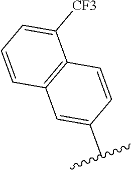 [SMILES: *c1ccc2c(C(F)(F)F)cccc2c1] | 4-Cl-phenyl |
| 24 | NH | 2 | 1 | 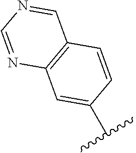 [SMILES: *c1ccc2cncnc2c1] | 4-CF₃-phenyl |
| 25 | NH | 1 | 2 | 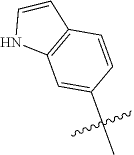 [SMILES: *c1ccc2cc[nH]c2c1] | 4-Et-phenyl |
| 26 | NH | 1 | 2 | 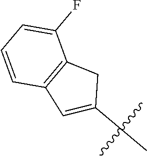 [SMILES: *C1=Cc2cccc(F)c2C1] | 2-F-4-Br-phenyl |
| 27 | NH | 1 | 1 | 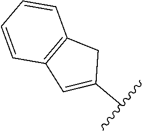 [SMILES: *C1=Cc2ccccc2C1] | 4-Br-phenyl |
| 28 | NH | 1 | 1 | 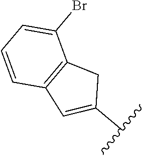 [SMILES: *C1=Cc2cccc(Br)c2C1] | 4-Br-phenyl |
| 29 | NH | 2 | 1 | 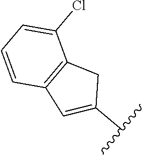 [SMILES: *C1=Cc2cccc(Cl)c2C1] | 4-Br-phenyl |
| 30 | NH | 1 | 2 | 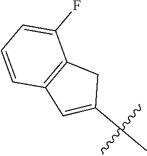 [SMILES: *C1=Cc2cccc(F)c2C1] | 4-Cl-phenyl |
| 31 | NH | 1 | 2 | 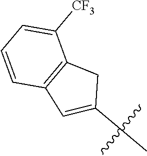 [SMILES: *C1=Cc2cccc(C(F)(F)F)c2C1] | 4-CF₃-phenyl |
| 32 | NH | 1 | 1 | 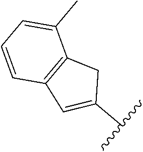 [SMILES: *C1=Cc2cccc(C)c2C1] | 4-Br-phenyl |
| 33 | NMe | 1 | 1 | 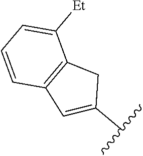 [SMILES: *C1=Cc2cccc(CC)c2C1] | 4-Br-phenyl |
| 34 | NH | 1 | 2 | 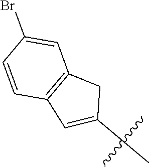 [SMILES: *C1=Cc2ccc(Br)cc2C1] | 4-Cl-phenyl |
| 35 | NH | 2 | 1 | 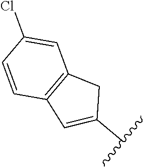 [SMILES: *C1=Cc2ccc(Cl)cc2C1] | 4-CF₃-phenyl |
| 36 | NH | 1 | 2 | 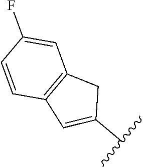 [SMILES: *C1=Cc2ccc(F)cc2C1] | 4-Et-phenyl |
| 37 | NH | 1 | 2 | 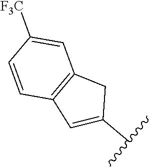 [SMILES: *C1=Cc2ccc(C(F)(F)F)cc2C1] | 2-F-4-Br-phenyl |
| 38 | NH | 1 | 1 | 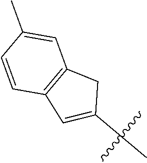 [SMILES: *C1=Cc2ccc(C)cc2C1] | 4-Br-phenyl |
| 39 | NH | 1 | 1 | 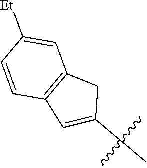 [SMILES: *C1=Cc2ccc(CC)cc2C1] | 4-Br-phenyl |
| 40 | NH | 1 | 1 | 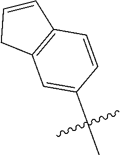 [SMILES: *c1ccc2c(c1)CC=C2] | 4-Br-phenyl |

In some embodiments, the compounds are represented by formula (I) and/or formula (IV).

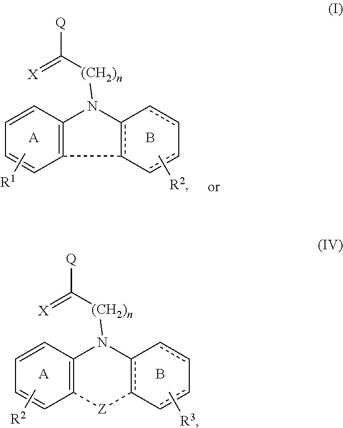

wherein
A is phenyl or a 6-membered heteroaromatic ring having 1, 2 or 3 N atoms in the heteroaromatic ring;
B is phenyl, or a 6-membered heteroaromatic ring having 1, 2 or 3 N atoms in the heteroaromatic ring; or a 6-membered aliphatic ring with up to 3 heteroatoms;
the dashed line between A and B indicates an optional bond;
R1 and R2 are independently none, C1-C5 alkyl, F, Cl, Br, I, CN, NO₂, NR4, NR4₂, OR4, CF₃, COOH, COOR4, NHR4, OCR4, OCOR4, OR4, SR4, SOR4, or SO₂R4;
X is H, NH₂, OH, O, F, Cl, Br, I, CN, SH, NO₂, NR4, NR4₂, OR, CF₃, COOH, R4, COOR4, NHR4, OCR4, OCOR4, OR4, SR4, SOR4, or SO₂R4; wherein the bond between X and the main scaffold is a single bond or a double bond, depending on the definition of X;
Q is

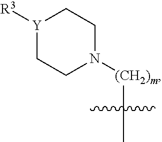

(CH₂)ₘN((CH₂)ₒR5)₂, COH, COOH, or CH₂NH(CH₂)₁OH
R3 is none, H, C1-C6 alkyl, R4(C═O), or (CH₂)ₚOH;
R4 is H or C1-C3 alkyl;
each R5 is independently OH, SH, NR4₂ or R4;
Y is O, S, N or CH;
Z is O, S, NR4, CHR4, S(O)₂, C(Me)₂ or C(O);
each l, m, n, o and p is independently 1-3;
or a pharmaceutically acceptable salt thereof.

In one embodiment of the methods and compounds disclosed herein, the compound can have, for example, the structure of formula (II), (III), (IV) or (VI)

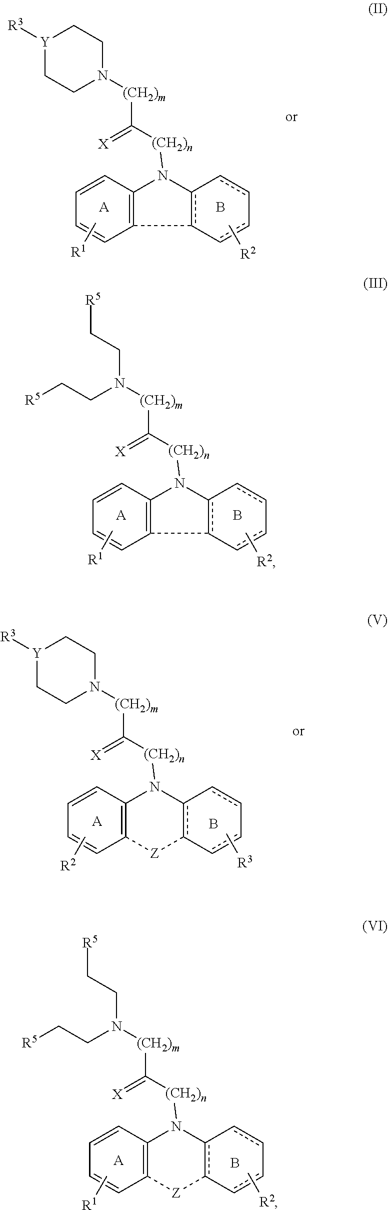

or a pharmaceutically acceptable salt thereof.

In one embodiment of the methods and compounds disclosed herein, there is no bond between A and B. In one embodiment, R1 and/or R2 are in the para position with respect to the bond to the N atom.

In one embodiment of the methods and compounds disclosed herein, the compound can have a structure, for example, selected from the group consisting of

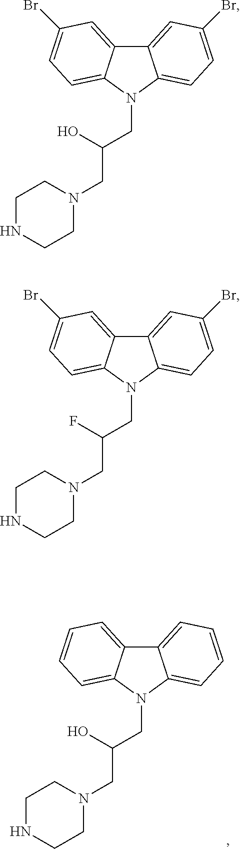 [SMILES: OC(CN1CCNCC1)Cn1c2ccc(Br)cc2c2cc(Br)ccc21 ; FC(CN1CCNCC1)Cn1c2ccc(Br)cc2c2cc(Br)ccc21 ; OC(CN1CCNCC1)Cn1c2ccccc2c2ccccc21]

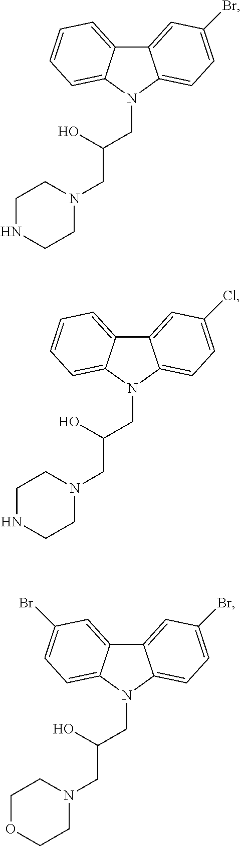 [SMILES: OC(CN1CCNCC1)Cn1c2ccccc2c2cc(Br)ccc21 ; OC(CN1CCNCC1)Cn1c2ccccc2c2cc(Cl)ccc21 ; OC(CN1CCOCC1)Cn1c2ccc(Br)cc2c2cc(Br)ccc21]

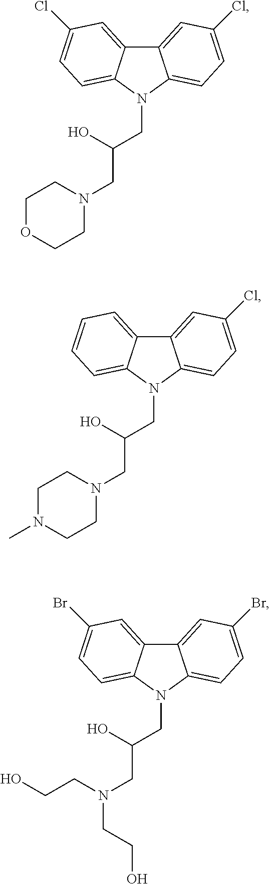 [SMILES: OC(CN1CCOCC1)Cn1c2ccc(Cl)cc2c2cc(Cl)ccc21 ; CN1CCN(CC(O)Cn2c3ccccc3c3cc(Cl)ccc32)CC1 ; OCCN(CCO)CC(O)Cn1c2ccc(Br)cc2c2cc(Br)ccc21]

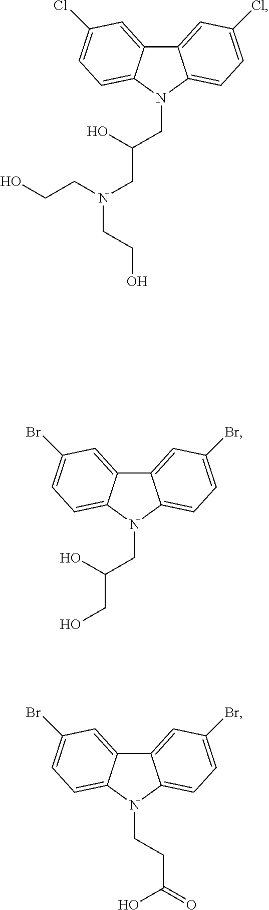 [SMILES: OCCN(CCO)CC(O)Cn1c2ccc(Cl)cc2c2cc(Cl)ccc21 ; OCC(O)Cn1c2ccc(Br)cc2c2cc(Br)ccc21 ; O=C(O)CCn1c2ccc(Br)cc2c2cc(Br)ccc21]

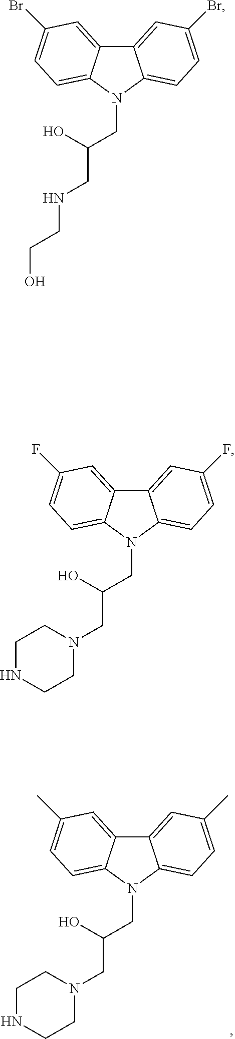 [SMILES: OCCNCC(O)Cn1c2ccc(Br)cc2c2cc(Br)ccc21 ; OC(CN1CCNCC1)Cn1c2ccc(F)cc2c2cc(F)ccc21 ; Cc1ccc2c(c1)c1cc(C)ccc1n2CC(O)CN1CCNCC1]

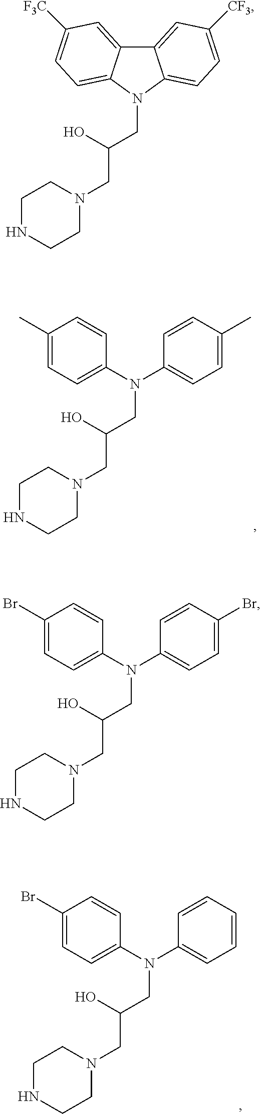 [SMILES: OC(CN1CCNCC1)Cn1c2ccc(C(F)(F)F)cc2c2cc(C(F)(F)F)ccc21 ; Cc1ccc(N(CC(O)CN2CCNCC2)c2ccc(C)cc2)cc1 ; OC(CN1CCNCC1)CN(c1ccc(Br)cc1)c1ccc(Br)cc1 ; OC(CN1CCNCC1)CN(c1ccccc1)c1ccc(Br)cc1]

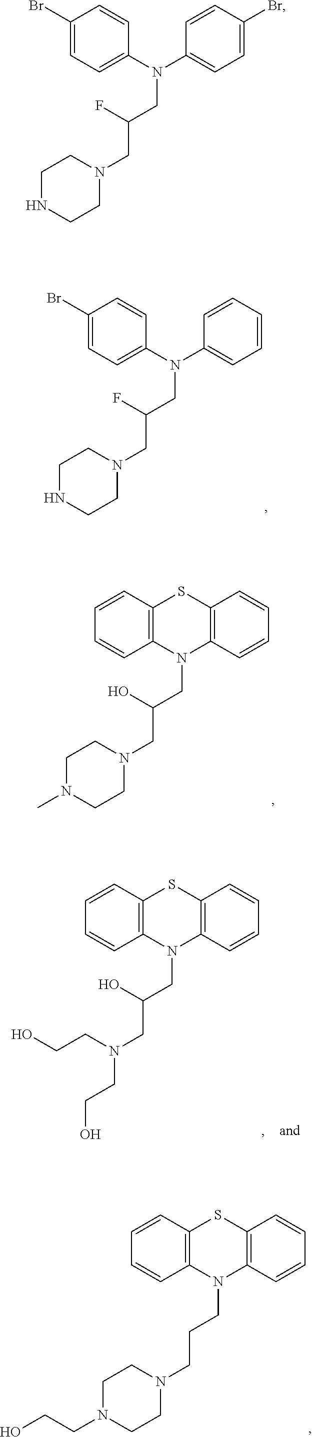 [SMILES: FC(CN1CCNCC1)CN(c1ccc(Br)cc1)c1ccc(Br)cc1 ; FC(CN1CCNCC1)CN(c1ccccc1)c1ccc(Br)cc1 ; CN1CCN(CC(O)CN2c3ccccc3Sc3ccccc32)CC1 ; OCCN(CCO)CC(O)CN1c2ccccc2Sc2ccccc21 ; OCCN1CCN(CCCN2c3ccccc3Sc3ccccc32)CC1]

or a pharmaceutically acceptable salt thereof.

Preferably, the one or more compounds is administered in an amount effective to inhibit BAX in a subject.

The invention also provides a method of inhibiting Bcl-2-associated x-protein (BAX) in a subject comprising contacting the BAX with one or more of the compounds of formula (I) and/or formula (IV) in an amount effective to inhibit BAX, wherein formula (I) and formula (IV) have the structure as defined herein.

Also provided is a method of inhibiting Bcl-2-associated x-protein (BAX) comprising contacting BAX with one or more of any of the compounds or pharmaceutical compositions disclosed herein in an amount effective to inhibit BAX. Preferably, the BAX is in a subject, and the one or more compounds or compositions is administered to the subject.

The subject being administered the compound, and being treated, may have, for example, a disease or condition is selected from the group consisting of hypoxic cardiomyocytes, cardiac ischemia, cardiac ischemia-reperfusion injury, myocardial infarction, myocardial infarction and reperfusion injury, chemotherapy-induced cardiotoxicity, arteriosclerosis, heart failure, heart transplantation, aneurism, chronic pulmonary disease, ischemic heart disease, hypertension, pulmonary hypertension, thrombosis, cardiomyopathy, stroke, a neurodegenerative disease or disorder, an immunological disorder, ischemia, ischemia-reperfusion injury, infertility, a hematological disorder, renal hypoxia, hepatitis, a liver disease, a kidney disease, an intestinal disease, liver ischemia, intestinal ischemia, asthma, AIDS, Alzheimer's disease, Frontotemporal Dementia, Taupathies, Parkinson's disease, Huntington's disease, retinitis pigmentosa, spinal muscular atrophy, cerebellar degeneration, amyotrophic lateral sclerosis, organ transplant rejection, arthritis, lupus, irritable bowel disease, Crohn's disease, asthma, multiple sclerosis, diabetes, premature menopause, ovarian failure, follicular atresia, fanconi anemia, aplastic anemia, thalassemia, congenital neutropenia, myelodysplasia, and a disease or disorder involving cell death and/or tissue damage.

In the case where the disease or condition is chemotherapy-induced cardiotoxicity, preferably the compound does not interfere with the ability of the chemotherapeutic agent to treat cancer. The chemotherapeutic agent can be, for example, one or more of doxorubicin and trastuzumab. The cancer can be, for example, one or more of a leukemia, a solid tumor, acute lymphoblastic leukemia (ALL), acute myeloid leukemia (AML), chronic lymphocytic leukemia (CLL), chronic myeloid leukemia (CML), breast cancer, prostate cancer, lymphoma, skin cancer, pancreatic cancer, colon cancer, melanoma, malignant melanoma, ovarian cancer, brain or spinal cord cancer, primary brain carcinoma, medulloblastoma, neuroblastoma, glioma, head-neck cancer, glioma, glioblastoma, liver cancer, bladder cancer, stomach cancer, kidney cancer, placental cancer, cancer of the gastrointestinal tract, non-small cell lung cancer (NSCLC), head or neck carcinoma, breast carcinoma, endocrine cancer, eye cancer, genitourinary cancer, cancer of the vulva, ovary, uterus or cervix, hematopoietic cancer, myeloma, leukemia, lymphoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, chronic granulocytic leukemia, acute granulocytic leukemia, acute myelogenous leukemia, chronic myelogenous leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft tissue cancer, soft-tissue sarcoma, osteogenic sarcoma, sarcoma, primary macroglobulinemia, central nervous system cancer and retinoblastoma.

In some embodiments, the disease is musculoskeletal disease or neurodegenerative diseases or retinal diseases or fibrosis associated with inflammation. In some embodiments, the inflammation is associated with osteoarthritis, osteoporosis, rheumatoid arthritis, lupus, gout, neurodegenerative diseases (Alzheimer's disease, Frontotemporal Dementia, Taupathies, Parkinson's disease, Huntington's disease, amyotrophic lateral sclerosis,) or neuroinflammatory disorders.

Also provided is a method of treating a myocardial infarction or a myocardial infarction and reperfusion injury in a subject comprising administering to the subject one or more of the compounds or pharmaceutical compositions disclosed herein in an amount effective to treat a myocardial infarction or a myocardial infarction and reperfusion injury in a subject in need thereof. Preferably, the one or more compounds or the pharmaceutical composition is administered in an amount effective to inhibit Bcl-2-associated x-protein (BAX) in a subject.

Also disclosed herein is a method of storing a biological material ex vivo or prolonging the viability of the biological material ex vivo for a period of time, comprising contacting the biological sample during the period with an effective amount of the compound disclosed herein or the pharmaceutically acceptable salt thereof or a composition thereof containing additionally at least one pharmaceutically acceptable carrier.

The method is capable of inhibiting cell death in a biological material ex vivo, for example, during cold storage and/or after a period of warm injury. The method may also include maintaining the population of mammalian cells or the mammalian tissue or organ in the composition substantially at a temperature, for example, ranging from about −30° C. to about 10° C., from about −20° C. to about 0° C., or from about −20° C. to about 30° C. for a certain period of time. During the period of contacting the sample with the composition or maintaining the sample within the composition, 60% or more of the the biological material remains viable. The sample can be submerged, partially or completely, in the composition, coated by the composition, or in contact with the composition in any manner suitable to maintain its viability for a desirable period of time.

In some embodiments, prior to the period of contacting the biological sample or maintaining it with the composition, there includes optionally a step of flushing, rinsing or treating it with the composition at the time of sample harvest. The amount of the compound or its salt for this optional step can be determined by the weight or nature of the sample. For instance, the amount of the compound can range from about 0.1 to about 100, from about 0.5 to about 50, from about 1 to about 20, or from about 2 to about 10 mg/kg of the weight of the biological material. Nonlimiting examples of the amount of the compound include about 0.1, about 0.5, about 1, about 2, about 5, about 10, about 15, or about 20 mg/kg of the weight of the biological material. This flushing or treatment step may be repeated by one, two or more times.

Using the compositions disclosed herein, the present methods now permit maintenance of biologic activity and/or cellular viability of explanted tissues even when stored under appropriate conditions for periods of time of from several days, to several weeks, and even up to and including several months post-harvest. In some embodiments, the amount of the composition or the compound of Formula I or a pharmaceutically acceptable salt thereof is selected so that the cell death is reduced by more than 10%, more than 20%, more than 30%, more than 40%, more than 50% or more than more than 60% in comparison with a reference without the composition over a period of about 6 hours, about 12 hours, about 24 hours, about 36 hours, about 48 hours, about 60 hours, or about 72 hours, or for a period of 1, 2, 5, 7, 10, 12, 14, 15, 30, 60, 90, 120, 150, or more days at a temperature of for example about −30° C., about −20° C., about −10° C., about −5° C., about 0° C., about 5° C., about 10° C., about 15° C., about 20° C. or about 25° C.

This method can be applied to storing and/or transporting one or more populations of mammalian cells, tissues, or organs, under conditions that permit long-term retention of viability and/or biological activity and/or function. The method is capable of keeping the population of mammalian cells or the mammalian tissue or organ viable both during storage and immediately thereafter maintaining such cells or tissues/organs in a physiologic state that is suitable for their implantation into a selected recipient mammal.

In some embodiments, the present methods permit retention of 60% or more (e.g. 65%, 70%, 75%, 80%, 85%, 90%, 95% or more) of the initial post-harvest viability of a biological material when stored in such compositions and maintained under appropriate environmental conditions over a period of about 6 hours, about 12 hours, about 24 hours, about 36 hours, about 48 hours, about 60 hours, or about 72 hours, or for a period of 1, 2, 5, 7, 10, 12, 14, 15, 30, 60, 90, 120, 150, or more days at a temperature of, for example, about −30° C., about −20° C., about −10° C., about −5° C., about 0° C., about 5° C., about 10° C., about 15° C., about 20° C. or about 25° C. In illustrative embodiments, various biological materials may be prepared, stored, and transported under conditions that permit the recovered cells, tissues, or organs to retain significant viability (e.g., 75%, 80%, 85%, 90%, or 95% of their initial post-harvest viability) when compared to storage of similar biological samples in conventional buffers, organ transport solutions, or mammalian growth media alone.

In some embodiments, more than about 60%, more than about 65%, more than about 70%, more than about 75%, more than about 80%, more than about 85%, more than about 90%, more than about 95% or more than about 98% of the biological material (e.g. a population of mammalian cells, tissue, organ, etc.) remains viable after maintaining the material in the composition over a period of about 6 hours, about 12 hours, about 24 hours, about 36 hours, about 48 hours, about 60 hours, or about 72 hours, or for a period of 1, 2, 5, 7, 10, 15, 30, 60, 90, 120, 150, or more days at a temperature of, for example, about −30° C., about −20° C., about −10° C., about −5° C., about 0° C., about 5° C., about 10° C., about 15° C., about 20° C. or about 25° C. In some embodiments, the biological remains substantially viable after maintaining the material in the composition over a period of about 6 hours, about 12 hours, about 24 hours, about 36 hours, about 48 hours, about 60 hours, about 72 hours, or longer than 80 hours. In those conditions, a biological material that is “substantially viable” includes, but is not limited to the organ, tissue or population of cells, that when stored, maintained, and/or transported in one or more of the compositions disclosed herein, that are at least about 95% viable, at least about 96% viable, at least about 97% viable, at least about 98% viable, or even at least about 99% viable.

It is also contemplated that the disclosed compositions will find particular utility in methods for preserving sufficient biological functional and retaining sufficient cellular viability and/or tissue integrity in poorly-perfusable mammalian tissues that have previously not been amenable to long-term storage. Through use of the present compositions, tissues that were previously only biologically-viable for implantation following short-term (e.g., several hours to several days) storage, may now be prepared that are substantially biologically active and amenable to intermediate-term (e.g., several days to several weeks) and even extended or long-term (e.g., several weeks to several months or more) storage. Such methods thereby significantly extend the conventional harvest-to-implantation “window of opportunity,” and provide novel methods for extending the usable “shelf-life” of recovered tissues or cultured cell populations from several hours to many weeks to even several months or longer.

While the methods and compositions of the present invention are contemplated to be useful in the storage and viability-preserving function of a variety of animal cells, tissues, and organs outside the living body of the donor, such methods and compositions are particularly suited for the harvest, storage and transport of mammalian cells, tissues, and organs. Especially relevant are those cells, tissues, or organs that are recovered from suitable living or cadaveric donor mammals and are destined for implantation into suitable mammalian recipients.

Exemplary types of mammalian cells which may be recovered, stored, and/or transported using one or more of the methods and compositions described herein include, but are not limited to: chondral cells, cartilagenous cells, osteochondral cells, islet cells, osteogenic cells, neural cells, bone cells, bone marrow cells, adipose cells, fibroblasts, muscle cells, blood, blood components, stem cells, and embryonic stem cells.

Exemplary types of mammalian tissues which may be recovered, stored, and/or transported according to the present invention include, but are not limited to, skin, cartilage, tendons, ligaments; fascia, tibialis, patellas and other bones, heart valves, semi-tendinous tissues, blood vessels, vertebral discs, corneas, lenses, meniscus, hair, adipose tissue, fibrous tissue, neural tissue, connective tissue, and striated, smooth, or cardiac muscle tissue. The cells or tissues may be recovered from human or animal subjects and are then processed and/or cryopreserved (frozen) for later implantation. Allograft tissues, including, but not limited to, heart valves and portions of heart valves, aortic roots, aortic walls, connective tissues including fascia and dura, vascular grafts (including arterial, venous, and biological tubes), and orthopedic soft tissues, such as boned-or non-boned tendons or ligaments, are often subjected to cryogenic preservation. In this manner, a ready supply of these valuable tissues can be made available for later implantation into mammals, especially humans. In addition, viable xenograft tissues from transgenic animals or tissues developed from human or non-human cells that may include differentiated cell types, stem cells, or genetically-modified cells of various origins may be appropriately processed, cryopreserved, and stored for later implantation. Additional examples include engineered cells of tissues or tissue engineered constructs.

Explanted animal tissues, cell populations, and recovered mammalian organs stored or maintained by any one of the methods or processes disclosed herein, or any explanted mammalian cells, tissue, or organ stored in one or more of the disclosed compositions are preferably suitable for implantation into a selected recipient animal, and particularly into a selected recipient mammal. Examples of mammalian species into which the explanted tissue may be transplanted, include, but are not limited to, humans, cattle, horses, sheep, pigs, goats, rabbits, dogs, cats, and non-human primates.

In some embodiment of any method disclosed herein, the cell types may include chondral, cartilagenous, osteochondral, islet, osteogenic, neural, bone, bone marrow, adipose, fibroblast, muscle, blood, and stem cells; the animal tissues may include skin, bone, cartilage, tendon, ligament, vertebral disc, cornea, lens, meniscus, hair, striated muscle, smooth muscle, cardiac muscle, adipose tissue, fibrous tissue, neural tissue, and connective tissue; or the mammalian organs may include cochlea, testis, ovary, stomach, lung, heart, liver, pancreas, kidney, intestine, and eye.

Cell populations, tissues and organs prepared by the processes provided herein may be of any origin, although those of animal origin and of mammalian origin in particular, are preferable. Exemplary explanted biological materials may be obtained from one or more animals, including, but not limited to, bovines, canines, caprines, equines, felines, gallines, humans, lapines, leporines, lupines, murines, ovines, porcines, vulpines, or non-human primates.

The compositions disclosed herein may also be used to perfuse the tissues, organs, or circulatory system of the donor animal prior to harvest (either while the animal is still alive, or alternatively, postmortem). The disclosed compositions may also be used as a wash solution to cleanse the freshly-recovered tissues from the host animal prior to long-term storage, transport, or transplantation.

In the practice of the present invention, it is often desirable to maintain the cells, tissues, or organs in the composition essentially from a time immediately post-harvest until the explant material is readied for transplantation into a recipient mammal. During the interval between harvest and implantation, it is also desirable to monitor and control the environmental conditions, and storage parameters to maintain the integrity, viability, and biochemical activity of the recovered biological material.

In some embodiments, the compositions or methods disclosed herein find particular use in the storage and/or transport of tissues at an ambient storage temperature in the range from about −55° C. to about 25° C., from about −45° C. to about 35° C., from about −25° C. to about 25° C., or from about −15° C. to about 15° C. Preferably, at least a portion of the storage solution remains substantially in an unfrozen, or liquid state. While it is contemplated that slight variation in temperature during the storage/transport process will not adversely affect the integrity, biological function, or cellular viability of the stored tissue or organ, in some embodiments the material is maintained and transported under environmental conditions of approximately −10° C. and about 25° C., or from between about −5° C. and about 20° C., from between about 0° C. and about 15° C., or from between about 0° C. and about 10° C. Nonlimiting examples of the temperature within the aforementioned ranges include about −30° C., about −20° C., about −10° C., about −5° C., about 0° C., about 5° C., about 10° C., and about 15° C.

To preserve the integrity and viability of the biological material to the best extent possible, it may be desirable to contact freshly-recovered cells, tissues, or organs with the disclosed tissue viability-preserving compositions/formulations substantially immediately upon harvest, and to maintain the recovered cells, tissues, or organs in these formulations substantially until immediately prior to implantation. Pre-cooling of the composition to the desired storage temperature prior to contacting it with the recovered cells or tissues will often be desirable.

The method disclosed herein enhances viability of the biological materials during cold storage either with or without a preceding period of warm ischemic injury. A preceding period of warm ischemic injury refers to a period of reduced or no blood flow within the organ or tissue between when life support is withdrawn from the donor and death is declared. The period may range from about 1 minute to about 24 hours, from about 1 hour to about 15 hours, from about 2 hours to about 10 hours, from about 1 minute to about 10 hours, from about 1 minute to about 3 hours, from about 1 minute to about 1 hour, from about 5 minute to about 40 minutes, from about 5 minute to about 20 minutes, or from about 10 minute to about 30 minutes.

In certain circumstances, it may also be desirable to irrigate, infuse, perfuse, or wash the recovered biological material with one or more portions of the compositions immediately upon removal from the living or cadaveric donor organism, and then to subsequently transfer the washed biological material to a fresh aliquot of the composition just prior to storage.

In some circumstances, depending upon the tissue type, and the length of storage, it may also be desirable to periodically decant the “spent” medium of the composition from the stored tissue, and to replenish the storage means with fresh medium. Likewise, it may also be desirable to perform one or more additional perfusion or wash steps after removing the tissue from storage, and immediately prior to implantation into the recipient animal.

In some embodiments of any method disclosed herein, there may also include a step of cryogenically-preserving (i.e., freezing) a population of cells, tissues, or organs using tissue preservative buffers, solutions, or supplemented growth media. The step of freezing the tissue or biological material may optionally include the addition of one or more cryoprotectants or cryopreservative compounds to further permit freezing of the sample, and/or maintenance of the sample at temperatures generally below 0° C. Exemplary cryoprotectants and/or cryopreservative compounds, as used in the context of the present invention may include, but are not limited to, ice-suppressing cryoprotectants (e.g., non-colligative agents such as Supercool X-1000™ and Supercool Z-1000™M, 21st Century Medicine, Rancho Cucamonga, Calif.) glycerol, dimethylsulfoxide (DMSO), ethylene glycol, propylene glycol, polyethylene oxide (PEO), acetamide, ethanol, methanol, butanediol, carbohydrates (including sugars such as glucose, fructose, dextrans, sucrose, lactose, and trehalose), polyvinyl alcohols, hydroxyethyl starch, serum albumin, and such like.

In some embodiments, it may also be desirable to provide one or more optional additional steps in method, including, for example, steps that involve freezing and/or thawing of a tissue sample or cell population. Such freezing and thawing steps may be achieved by any conventional manner known to those in the art, (e.g., slowly bringing the temperature of a refrigerated tissue or cell sample down to a suitable sub-zero temperature, or alternatively, slowly bringing the temperature of a sub-zero stored sample up to refrigerated (and, optionally, to either room or recipient body temperature immediately prior to implantation). Such additional steps in the method may employ submersion vessels or frozen storage means to prepare the frozen tissue or cell sample, while conventional means such as a heated water bath or such like device, submerging the frozen sample directly into a sample of growth medium, biological buffer, or tissue/organ storage solution (e.g., pre-warmed to the desired temperature), may be employed to bring the temperature of a frozen tissue sample to the desired temperature required for transplanting the biological material into the body of a suitable recipient animal. Additional examples on freezing and thawing are available in U.S. Pat. No. 7,129,035, the entire disclosure of which is hereby incorporated by reference.

In some embodiments of any method disclosed herein, the subject can be, for example, a mammal, and is preferably a human.

As used herein, “treating” or to “treat” a disease or disorder means to alleviate or ameliorate or eliminate a sign or symptom of the disease or disorder that is being treated. When the compound or composition is administered to a subject before or at the onset of a disease or disorder, the compound or composition can prevent or reduce the severity of the disease or disorder. For example, administration of the compound to a subject can prevent or reduce the severity of chemotherapy-induced cardiotoxicity that would occur in the absence of administration of the compound. Administration of the compound can include preventive and/or therapeutic administration.

The compounds and compositions of the present invention can be administered to subjects using routes of administration known in the art. The administration can be systemic or localized to a specific site. Routes of administration include, but are not limited to, intravenous, intramuscular, intrathecal or subcutaneous injection, oral or rectal administration, and injection into a specific site.

Preferably, the compounds and compositions disclosed herein are administered acutely to treat a disease or disorder, due to potential hazards of long-term inhibition of cell death, e.g. cancer. The therapy can be used in conjunction with effective existing therapies for treating the disease or disorder, such as, e.g., angioplasty/stenting for cardiovascular disease.

In some embodiments, the compounds disclosed herein is represented by a the structure of formula (VII)

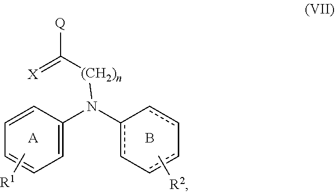

wherein
A is phenyl or a 6-membered heteroaromatic ring having 1, 2 or 3 N atoms in the heteroaromatic ring;
B is phenyl, or a 6-membered heteroaromatic ring having 1, 2 or 3 N atoms in the heteroaromatic ring; or a 6-membered aliphatic ring with up to 3 heteroatoms;
R1 and R2 are independently none, C1-C5 alkyl, F, Cl, Br, I, CN, NO₂, NR4, NR4₂, OR4, CF3, COOH, COOR4, NHR4, OCR4, OCOR4, OR4, SR4, SOR4, or SO₂R4;
X is H, NH₂, OH, O, F, Cl, Br, I, CN, SH, NO₂, NR4, NR4₂, OR, CF₃, COOH, R4, COOR4, NHR4, OCR4, OCOR4, OR4, SR4, SOR4, or SO₂R4; wherein the bond between X and the main scaffold is a single bond or a double bond, depending on the definition of X;
Q is

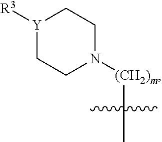

(CH₂)ₘN((CH₂)ₒR5)₂, COH, COOH, or CH₂NH(CH₂)₁OH;
R3 is none, H, C1-C6 alkyl, R4(C═O), or (CH₂)ₚOH;
R4 is H or C1-C3 alkyl;
each R5 is independently OH, SH, NR4₂ or R4;
Y is O, S, N or CH;
each l, m, n, o and p is independently 1-3;
or a pharmaceutically acceptable salt thereof.

In some embodiments, the compounds disclosed herein is represented by a the structure of formula (VIII) or (IX)

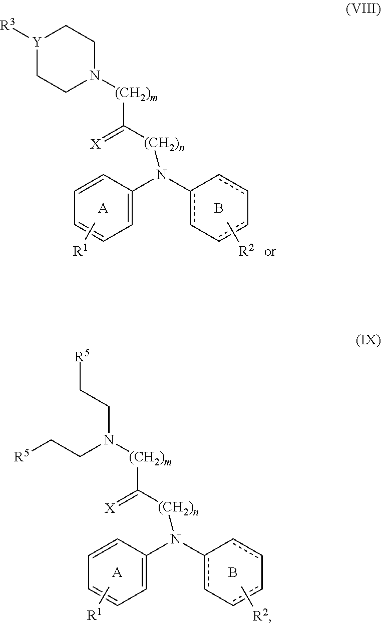

or a pharmaceutically acceptable salt thereof.

The compound can have, for example, a structure selected from the group consisting of

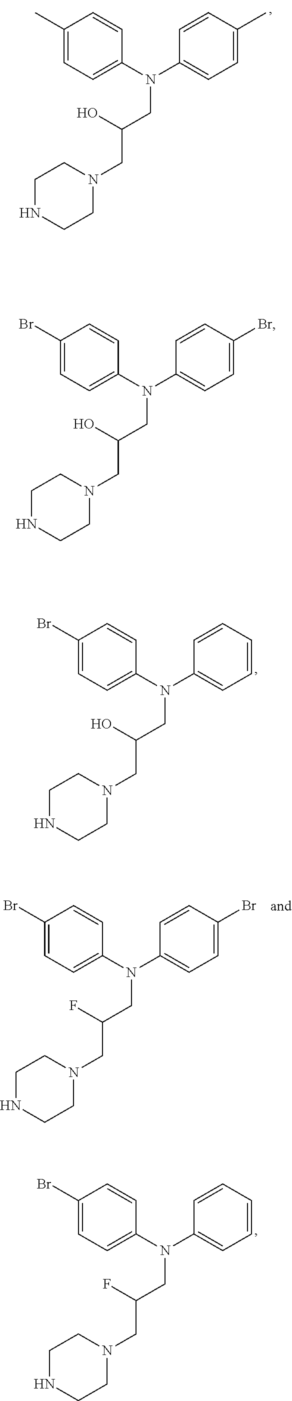 [SMILES: Cc1ccc(N(CC(O)CN2CCNCC2)c2ccc(C)cc2)cc1 ; OC(CN1CCNCC1)CN(c1ccc(Br)cc1)c1ccc(Br)cc1 ; OC(CN1CCNCC1)CN(c1ccccc1)c1ccc(Br)cc1 ; FC(CN1CCNCC1)CN(c1ccc(Br)cc1)c1ccc(Br)cc1 ; FC(CN1CCNCC1)CN(c1ccccc1)c1ccc(Br)cc1]

or a pharmaceutically acceptable salt thereof.

In some embodiments, the compounds disclosed herein is represented by a the structure of formula (X) or (XI)

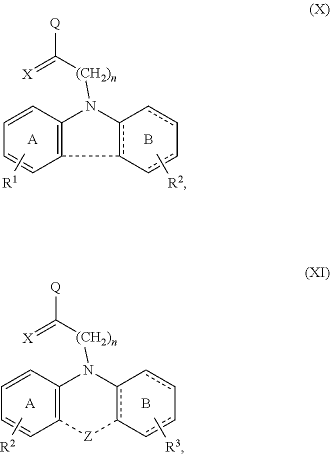

wherein
A is phenyl or a 6-membered heteroaromatic ring having 1, 2 or 3 N atoms in the heteroaromatic ring;
B is phenyl, or a 6-membered heteroaromatic ring having 1, 2 or 3 N atoms in the heteroaromatic ring; or a 6-membered aliphatic ring with up to 3 heteroatoms;
with the proviso that at least one of A and B is not phenyl;
the dashed line between A and B indicates an optional bond;
R1 and R2 are independently none, C1-C5 alkyl, F, Cl, Br, I, CN, NO₂, NR4, NR4₂, OR4, CF₃, COOH, COOR4, NHR4, OCR4, OCOR4, OR4, SR4, SOR4, or SO₂R4;
X is H, NH₂, OH, O, F, Cl, Br, I, CN, SH, NO₂, NR4, NR4₂, OR, CF₃, COOH, R4, COOR4, NHR4, OCR4, OCOR4, OR4, SR4, SOR4, or SO₂R4; wherein the bond between X and the main scaffold is a single bond or a double bond, depending on the definition of X;

Q is

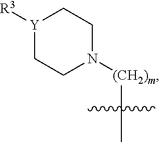

(CH₂)ₘN((CH₂)ₒR5)₂, COH, COOH, or CH₂NH(CH₂)₁H;
R3 is none, H, C1-C6 alkyl, R4(C═), or (CH₂)ₚOH;
R4 is H or C1-C3 alkyl;
each R5 is independently OH, SH, NR4₂ or R4;
Y is O, S, N or CH;
Z is O, S, NR4, CHR4, S(O)₂, C(Me)₂ or C(O);
each l, m, n, o and p is independently 1-3;
or a pharmaceutically acceptable salt thereof.

In one embodiment, Z is O, NR4, CHR4, S(O)₂, C(Me)₂ or C(O).

Pharmaceutically acceptable salts that can be used with compounds of the present invention include, e.g., non-toxic salts derived, for example, from inorganic or organic acids including, but not limited to, salts derived from hydrochloric, sulfuric, phosphoric, acetic, lactic, fumaric, succinic, tartaric, gluconic, citric, methanesulphonic and p-toluenesulphonic acids.

The invention also provides a pharmaceutical composition comprising one or more of the compounds disclosed herein and a pharmaceutically acceptable carrier. Pharmaceutically acceptable carriers and diluents that can be used herewith encompasses any of the standard pharmaceutical carriers or diluents, such as, for example, a sterile isotonic saline, phosphate buffered saline solution, water, and emulsions, such as an oil/water or water/oil emulsions. The pharmaceutical compositions can be formulated to be advantageous for the selected route of administration to a subject.

As used herein, “BAX” is Bcl-2-associated x-protein. In an embodiment, the BAX is mammalian. In a preferred embodiment, the BAX is a human BAX. In an embodiment, the BAX comprises consecutive amino acid residues having the following sequence:

(SEQ ID NO: 1)
MDGSGEQPRGGGPTSSEQIMKTGALLLQGFIQDRAGRMGGEAPELALDPV
PQDASTKKLSECLKRIGDELDSNMELQRMIAAVDTDSPREVFFRVAADMF
SDGNFNWGRVVALFYFASKLVLKALCTKVPELIRTIMGWTLDFLRERLLG
WIQDQGGWDGLLSYFGTPTWQTVTIFVAGVLTASLTIWKKMG.

As used herein, small molecule BAX inhibitors are defined as compounds that bind to BAX and inhibit its function.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### BAX is a Therapeutic Target for MI/R

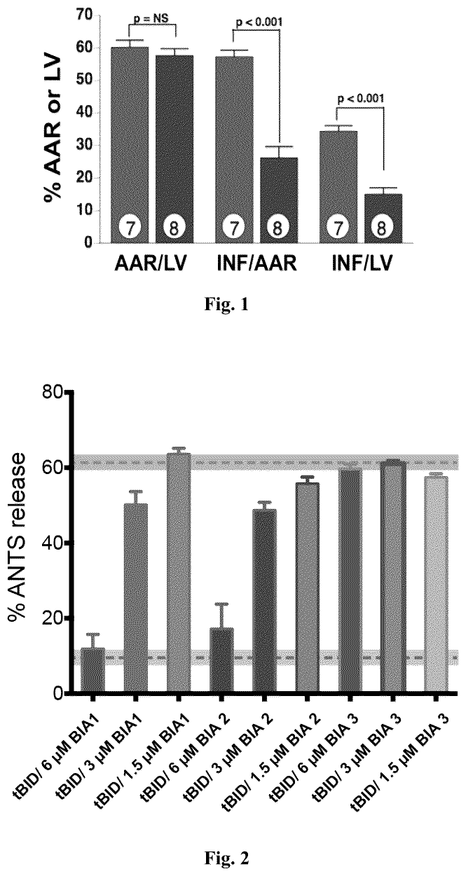

To determine whether BAX provides a therapeutic target for MI/R in vivo, wild type and BAX knockout (KO) mice were subjected to 45 min ischemia/24 h reperfusion. These KO mice have a generalized deletion of BAX making them a good model for the antagonism of BAX in both cardiomyocytes and non-myocytes. Area at risk (AAR) was measured by Evans blue dye and infarct size by tetrazolium chloride (TTC) staining (FIG. 1). Consistent with a previous study in isolated hearts (18), BAX deletion markedly reduced infarcts indicating that BAX plays an important role in the pathogenesis of infarction in vivo. These KO mice have a generalized deletion of BAX making them a good model for the antagonism of BAX in both cardiomyocytes and non-myocytes.

### Identification of a Small Molecule Inhibitor of BAX

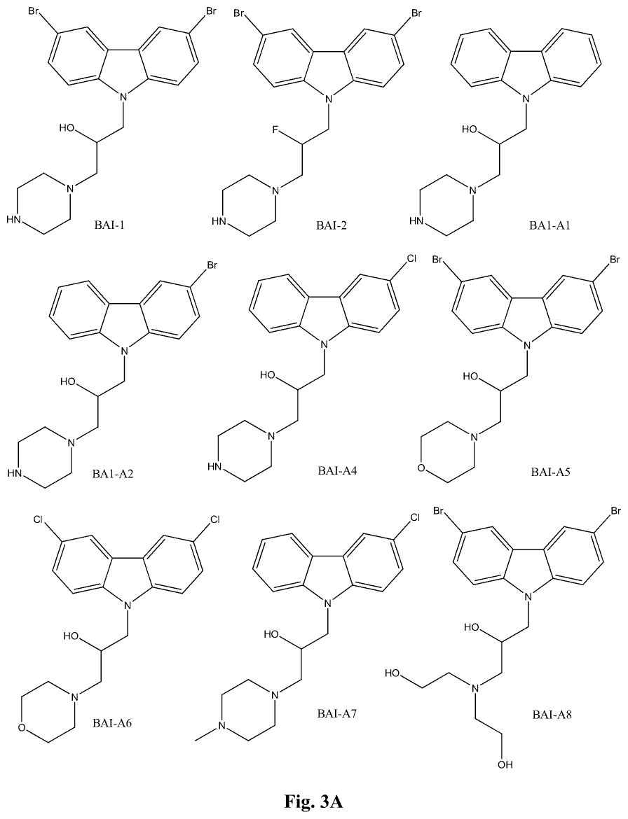

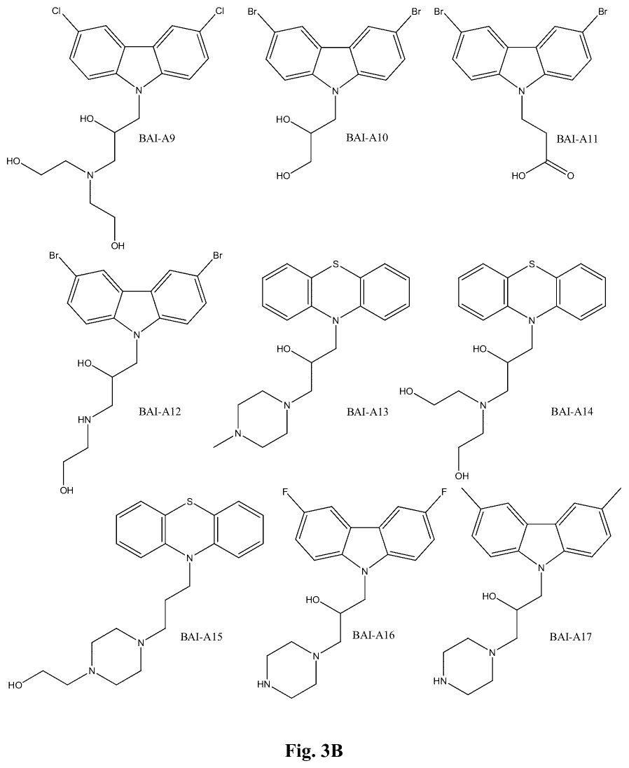

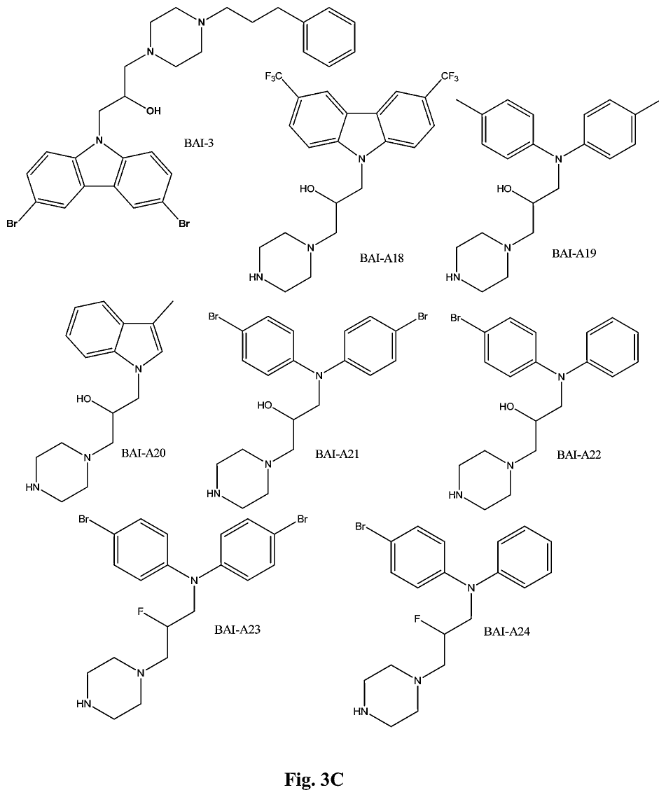

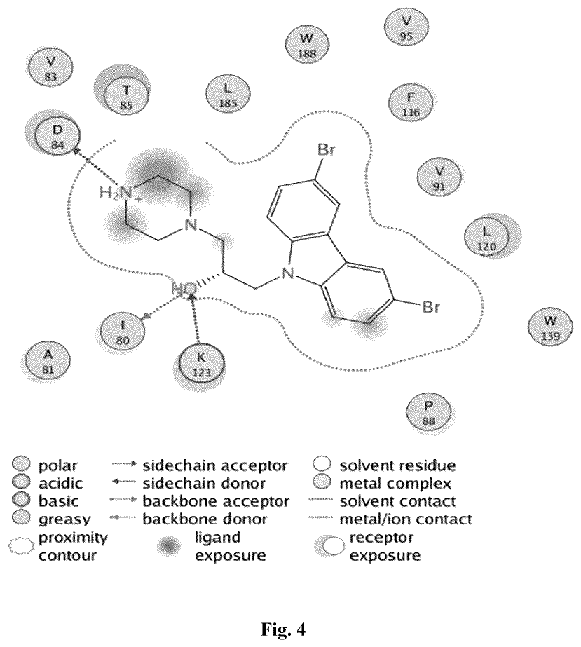

Previously, a small molecule screen using isolated mitochondria, revealed compounds that inhibit tBID-induced cytochrome c release. These compounds were hypothesized, but never shown, to work through BAX inhibition (19, 20). Accordingly, it was first investigated whether several of these small molecules inhibit BAX-mediated permeabilization of artificial membranes of similar lipid composition to the outer mitochondrial membrane (OMM). A liposome release assay (21), in which liposomes that contain a fluorophore are created, was used. Incorporation of tBID-activated BAX into the liposome membrane stimulates release of the fluorophore, providing a system to study BAX-mediated membrane permeabilization in isolation of other mitochondrial and cellular factors. Using this assay, a lead small molecule, termed BAX Activation Inhibitor 1 (BAI-1) (FIG. 3A), was shown to inhibit liposomal release of fluorophore in a dose-dependent and BAX-dependent manner (FIG. 2). Moreover, another compound, BAI-2, inhibited BAX-induced liposomal release (FIG. 2). These results indicate that inhibition of tBID-induced cytochrome c release from mitochondria by BAI-1 involves antagonism of BAX.

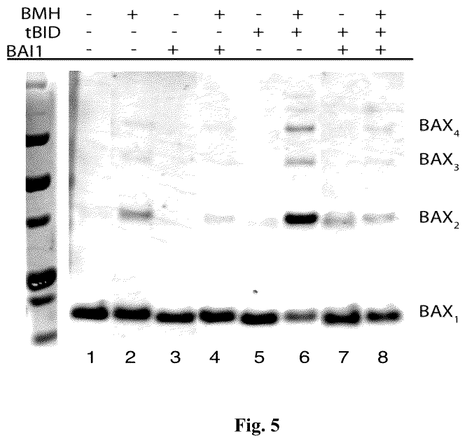

Next, it was tested whether BAI-1 binds to recombinant, purified BAX using 1H-NMR, and this was found to be the case (data not shown). To identify the mechanism of BAI-1 binding to BAX, ¹⁵N-¹H HSQC NMR analysis of BAX was performed upon titration of BAI-1. BAI-1 induced significant chemical shift perturbations on HSQC spectra of BAX, which were localized in the region formed by α-helices 3, 4, and 5 and the loop between α-helices 3 and 4 (data not shown). A few chemical shift changes in other regions of the structure were not localized and occurred predominantly in hydrophobic residues at the hydrophobic core of the BAX structure. These NMR data highlight a binding site for BAI-1 in a region of the BAX structure distinct from the trigger site and for which information does not currently exist regarding effects on BAX activation. To identify precisely how BAI-1 binds to and inhibits BAX, NMR data were used to guide molecular docking studies. FIG. 5 shows a close-up view of the novel BAX binding site and the bound docked structure of BAI-1.

It was hypothesized that BAI-1 stabilizes the interactions among these helices and the BAX structure and, through this mechanism, inhibits BAX conformational activation by BH3-only proteins. To assess inhibition of BAX activation by BAI-1, BAX oligomerization (which is downstream of activation) was tested using immunoblotting after cross-linking with BMH (FIG. 5). BAI-1 inhibited tBID-induced BAX oligomerization (lane 8 versus lane 6). Since BAI-1 does not inhibit the binding of BH3-only activator proteins to BAX (not shown), the most likely model is that BAI-1 functions as an allosteric inhibitor of BAX activation.

### BAI-1 Mechanism of Action

A key event in BAX activation is exposure of α helix 9 containing a transmembrane domain that inserts tightly into the outer mitochondrial membrane (OMM). BAX insertion into the OMM can be assessed by treating isolated mitochondria with strong alkali, which separates loosely attached proteins from mitochondria but fails to extract membrane-inserted proteins. Staurosporine (STS) treatment of mouse embryonic fibroblasts resulted in a pool of BAX that could not be retrieved by treatment of isolated mitochondria with strong alkali. Treatment of cells with BAI-1 significantly decreased the inserted pool. These data indicate that BAI-1 inhibits STS-induced exposure of BAX α helix 9. One important result of BAX conformational activation is its translocation from cytosol to mitochondria. Thus, it was also evaluated whether BAI-1 can inhibit STS-induced BAX translocation. Translocation was assessed by immunostaining for mitochondrial BAX puncta. BAI-1 significantly decreased BAX translocation to the mitochondria in a dose-dependent fashion. A similar result was observed with BAI-A22. (Data not shown.)

### BAI-1 Inhibits Apoptotic Cell Death and Necrotic Cell Death

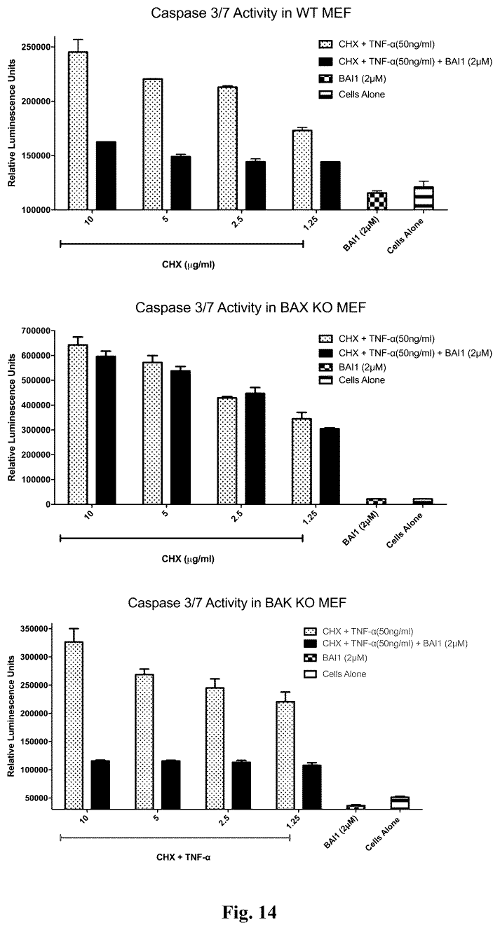

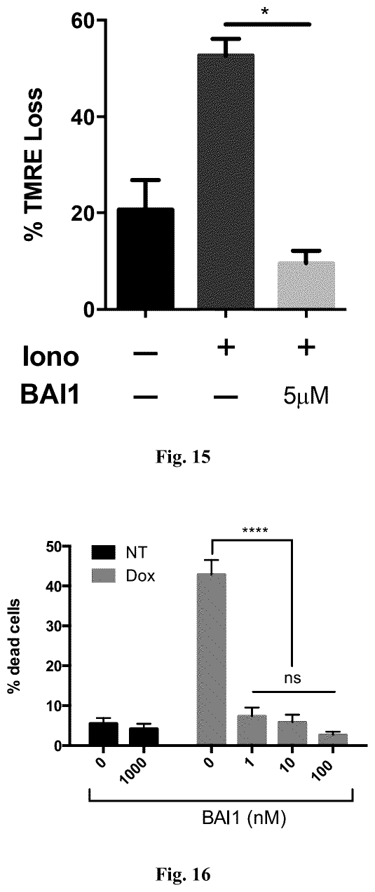

BAI-1 inhibits TNFα-induced BAX-mediated apoptotic cell death in mouse embryonic fibroblasts (FIG. 14). Inhibition of BAI-1 required BAX but not BAK. Nuclear fragmentation was also inhibited, as shown using BAI-1 and BAI-A22; externalization of phosphatidylserine was reduced as shown using BAI-1 (data not shown). BAI-1 also inhibits Ionomycin-induced BAX-mediated necrotic cell death in mouse embryonic fibroblasts as measured by the loss of TMRE fluorescence using FACS analysis (FIG. 15).

### BAI-1 Inhibits Cardiomyocyte Death

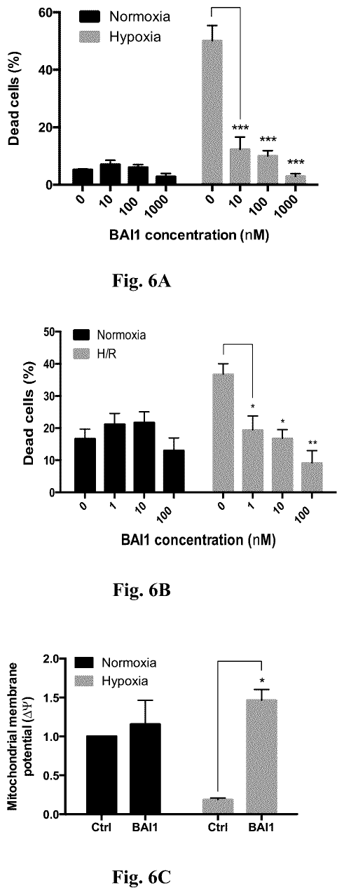

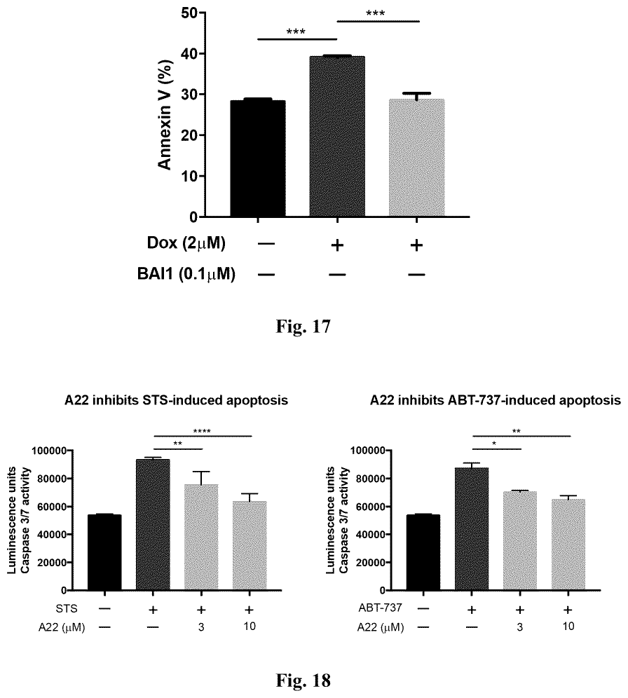

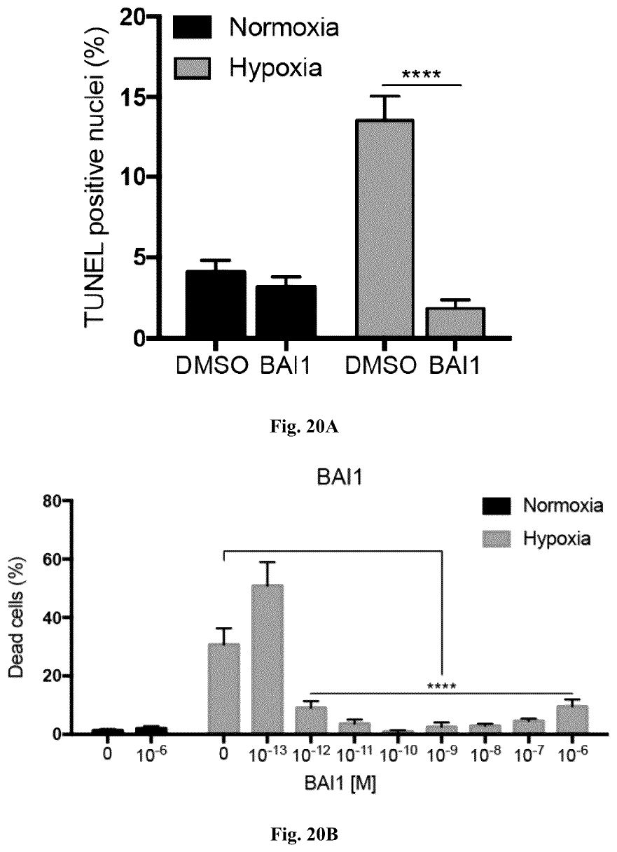

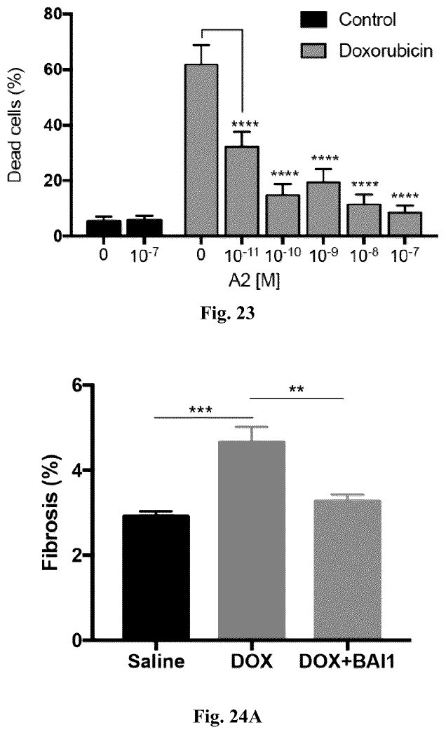

It was tested if BAI-1 can inhibit cell death in cardiomyocytes challenged with a noxious stimuli relevant to MI/R. At ˜10 nM concentrations, BAI-1 inhibited cell death in neonatal and adult cardiomyocytes challenged with 18 h of 3% hypoxia and 1 h hypoxia/2 h reoxygenation, respectively, both effects occurring in a dose-dependent manner (FIG. 6A,B). To explore the mechanism by which BAI-1 inhibits cardiomyocyte death, TMRM staining was used to assess its effect on loss of inner mitochondrial membrane potential (Δψm), which occurs with opening of the mPTP. BAI-1 markedly inhibited loss of Δψm (FIG. 6C). BAI-1 also inhibits tBID-induced BAX mediated cytochrome c release from isolated cardiac mitochondria. The levels of cytochrome c were quantified in mitochondrial and soluble fractions after incubation of mitochondria with tBID, BAX without and with BAI-1 (FIG. 13). It was also demonstrated that BAI-1 inhibits doxorubicin-induced cell death in rat neonetal cardiomyocytes (FIG. 16) and that BAI-1 inhibits doxorubicin-induced cell death in human cardiomyocytes derived from human induced pluripotent stem cells (FIG. 17). These data demonstrate that BAI-1 potently inhibits neonatal and adult cardiomyocyte death and that mitochondrial mechanisms are involved. BAI-1 inhibits cardiomyocyte apoptosis and necrotic cell death (FIG. 20A-B). BAI-A2 was also tested and shown to inhibit doxorubicin-induced cardiomyocyte necrosis (FIG. 23).

### BAI-Al Inhibits BAX During MI/R In Vivo

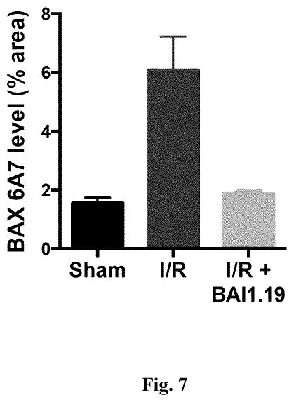

While BAI-1 potently inhibited cell death in isolated cardiomyocytes, it was discovered that BAI-1 is 99.9% bound to plasma proteins in mouse plasma (not shown). Based on its chemical structure, BAI-1 was re-engineered by removing both bromines to lower its hydrophobicity and, thereby, reduce its plasma protein binding. The resulting BAI-A1 (FIG. 3A) exhibited 89.4% plasma protein binding. As expected from the hypothesized role of the bromines in the BAI-1-BAX interaction, BAI-A1 exhibited less BAX binding than BAI-1 (not shown). Despite this, its ability to protect neonatal and adult cardiomyocytes against hypoxia and hypoxia/reoxygenation was only modestly decreased (not shown). Accordingly, the ability of BAI-A1 to inhibit BAX activation during MI/R was tested in vivo (FIG. 7). BAI-A1 potently blocks BAX conformational activation as assessed by immunostaining with 6A7, an antibody that recognizes only the active conformer of BAX. This was assessed following 45 min ischemia/6 h reperfusion, the time point that BAX is activated maximally in the absence of drug. It was concluded that BAI-A1 can access myocardial cells and is effective at inhibiting BAX activation during MI/R in vivo. The pharmacokinetics of BAI-A1 were measured in the mouse by injecting 1 mg/kg into a cohort of mice and harvesting plasma and heart tissue 0, 1, 6, and 24 h. The half-life of BAI-A1 was ˜3.5 h in both plasma and heart tissue.

Studies with Additional Compounds

FIG. 8 and Table 1 show IC50 values for BAI compounds tested in a BAX inhibition assay using tBID-induced BAX activation in liposomal membranes. The structures of the compounds are shown in FIG. 3.

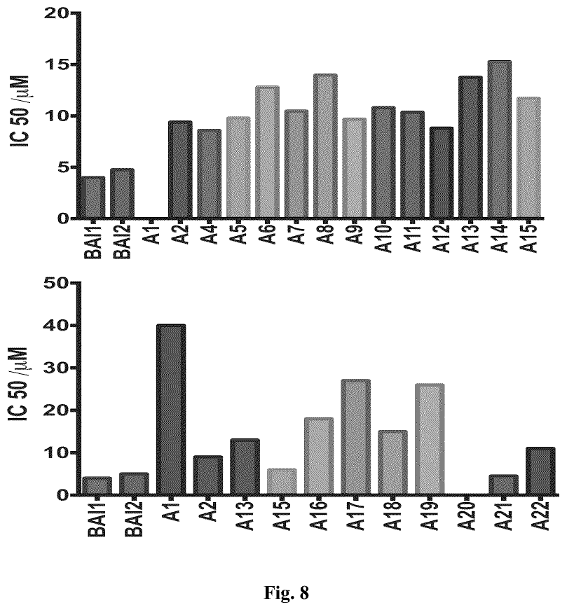

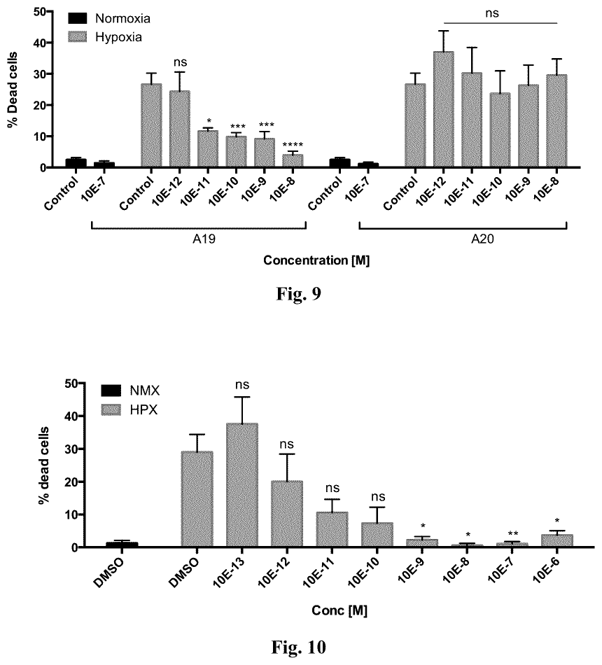

FIG. 9 shows that BAI-A19 potently inhibits primary neonatal cardiomyocyte death induced by hypoxia, whereas the inactive analog, BAI-A20, does not.

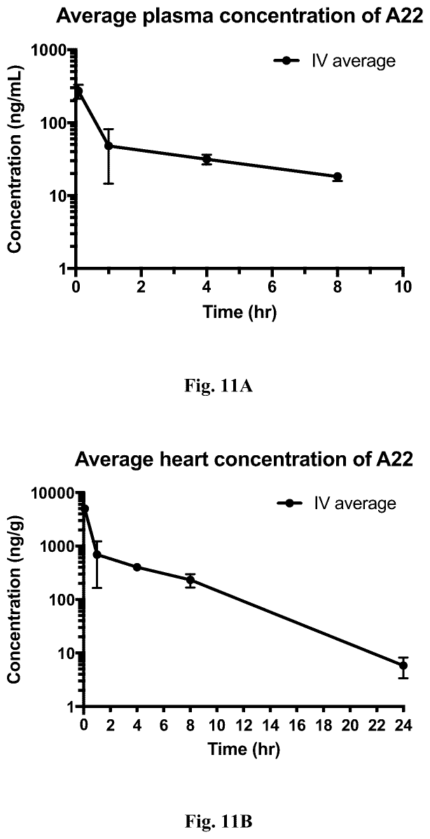

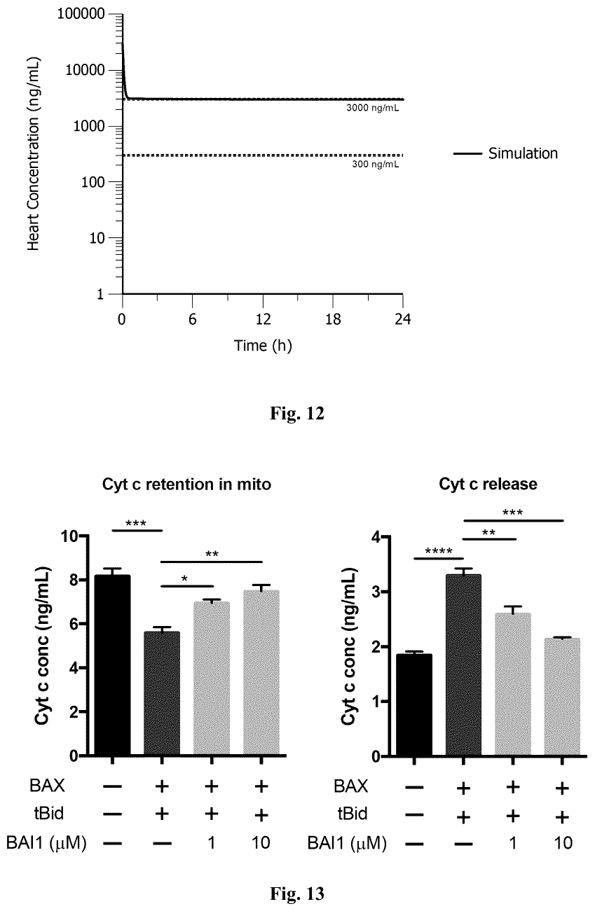

FIG. 18 shows that BAI-A22 inhibits doxorubicin-induced apoptosis in mouse embryonic fibroblasts. FIG. 10 shows that BAI-A22 potently inhibits primary neonatal cardiomyocyte death induced by hypoxia. FIG. 11 shows BAI-A22 pharmacokinetics in blood plasma and heart tissue. Pharmacokinetic parameters for BAI-A22 are shown in Table 2. Plasma protein binding was measured in rats for BAI-A22, which was found to be 95.6% bound. FIG. 12 shows the simulated heart concentration of BAI-A22.

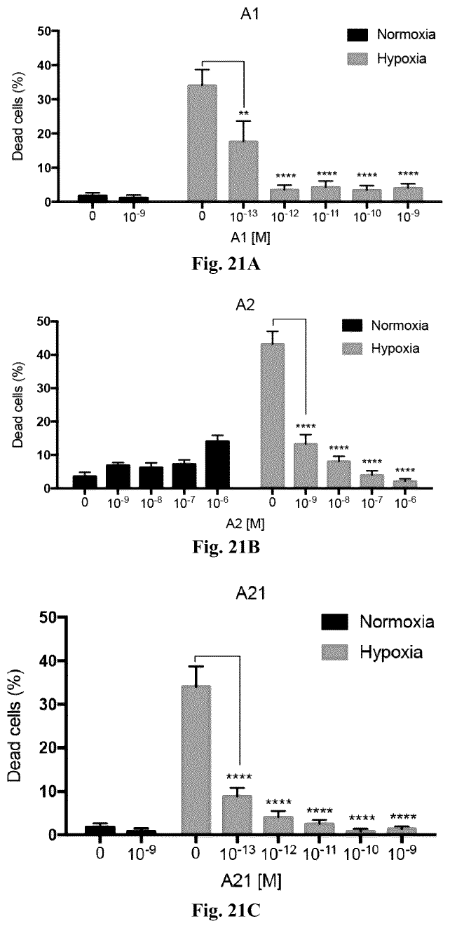

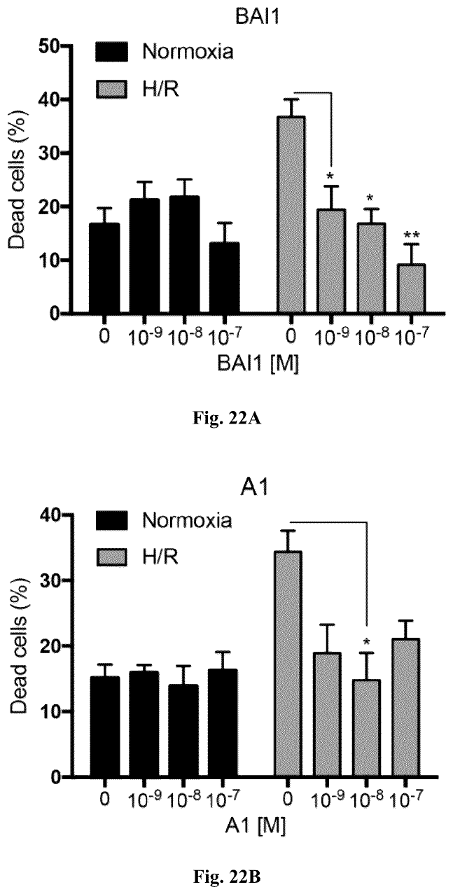

Compounds BAI-1 (FIG. 22A), BAI-A1 (FIGS. 21A, 22B), BAI-A2 (FIG. 21B) and BAI-A21 (FIG. 21C) inhibit necrosis in cardiomyocytes in a dose-dependent manner.

Pharmacokinetic properties were determined for compounds BAI-1, BAI-A1 and BAI-A22. Their half-life in plasma was, respectively, 45 hours, 3.5 hours and 5 hours.

### In Vivo Studies

### Effects of BAX Inhibitors on Chemotherapy-Induced Cardiotoxicity.

Doxorubicin is extensively used for both adults and children to treat many types of cancers, including solid tumors, such as breast cancer, leukemia and lymphomas (27). It is considered as one of the most potent of the Food and Drug Administration (FDA)-approved cancer drugs (28). Doxorubicin's clinical use is limited by its severe dose-dependent and often lethal heart failure (27), even emerging years after termination of treatment (29).

Studies using BAX knockout mice showed that deletion of Bax protected mice from doxorubicin-induced cardiac dysfunction as measured by improvements in fractional shortening and systolic wall thickening and decreases in apoptotic and necrotic cardiac cell death (data not shown).

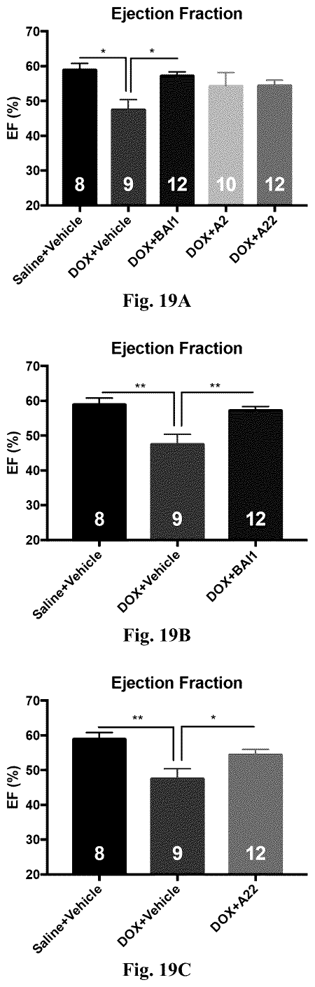

Doxorubicin-induced cardiotoxicity is known to increase BAX levels and drive apoptosis in cardiac cells and is a real clinical problem. A number of new chemotherapy drugs have the same side-effect and cardiotoxicity prevents their use at more effective doses. FIG. 19A-C illustrates the effects of three BAX inhibitors on heart function in doxorubicin-treated mice. C57BL/6 male mice 8 weeks of age were purchased from Charles River Laboratories and randomly grouped (n=8 to 13). An acute heart failure model was generated by a single intraperitoneal injection of doxorubicin (20 mg/kg) dissolved in saline. BAI-1, BAI-A2 and BAI-A22 were administered to mice by a single intraperitoneal injection of 2 mg/kg dose. Both BAI-1 (FIG. 19B) and BAI-22 (FIG. 19C) were therapeutically effective at the dose used. Similar therapeutic effects were observed with BAI-21 (data not shown).

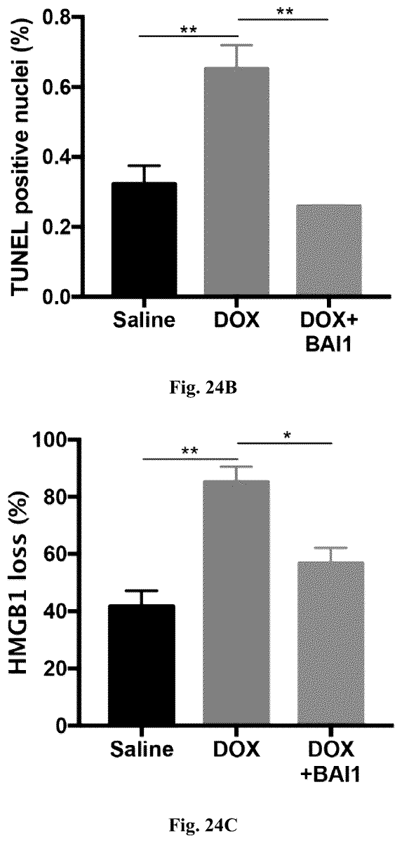

Hearts were collected from mice in the acute doxorubicin model and sectioned to measure fibrosis using Masson Trichrome staining, or stained for the apoptosis marker, TUNEL, or immunostained for HMGB1, loss of which indicates necrosis. BAI-1 reduced doxorubicin-induced cardiac fibrosis, apoptosis and cardiac necrosis (FIG. 24A-C).

### Chronic Doxorubicin-Induced Cardiomyopathy Mouse Model

Patients typically receive several “cycles” of doxorubicin administered at lower doses. The exact protocol depends on the cancer being treated. For example, some leukemias are treated with 4 cycles of 60 mg/m² (which would be the equivalent of 1.5 mg/kg) IV administered every 21-28 days. Cumulative doxorubicin dose of 20-25 mg/kg has been shown to induce a clinically relevant cardiomyopathy in mice (30-32). Based on this experience, a chronic protocol was used in which mice receive 3 mg/kg doxorubicin IP every other day ×8 doses (i.e. over a two-week period) for a cumulative dose of 24 mg/kg.

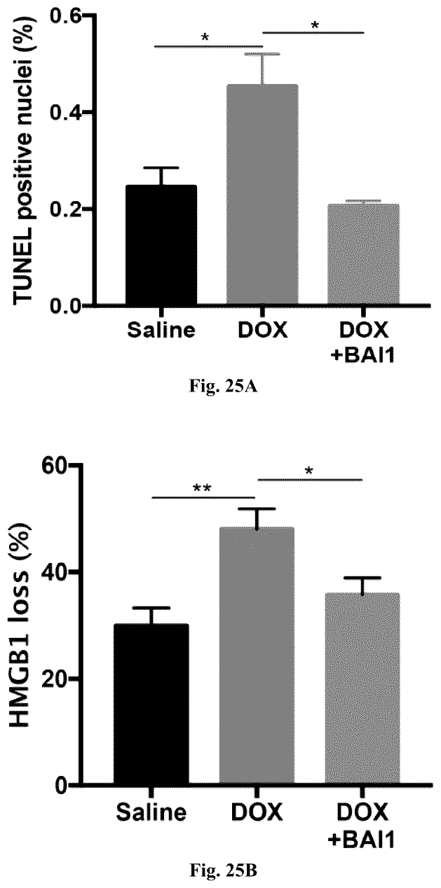

BAI-1 and BAI-A22 were tested for their ability to protect the heart against doxorubicin-induced cardiomyopathy, using a dose of 2 mg/kg. BAI1 and A22 significantly protected the heart from cardiac dysfunction as assessed by fractional shortening, ejection fraction and systolic wall thickening. Doxorubicin-induced apoptotic and necrotic cardiac cell death were largely abrogated as tested using BAI-1, as shown by TUNEL (FIG. 25A) and HMGB1 loss (FIG. 25B), respectively.

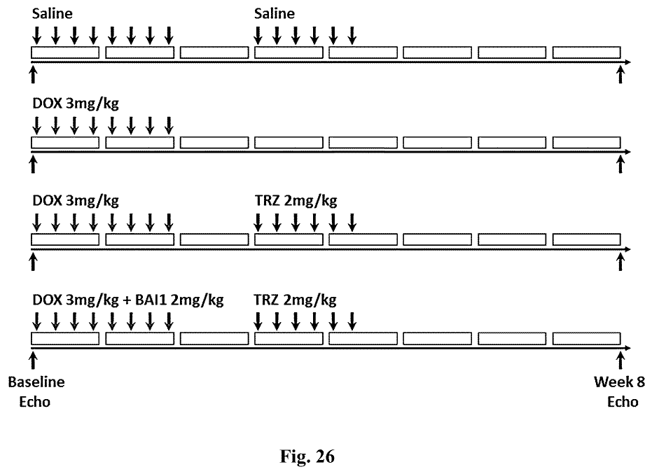

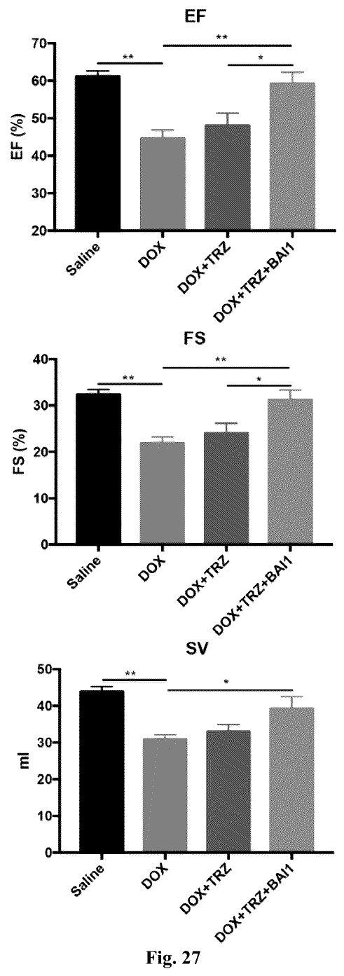

Trastuzumab is a humanized monoclonal antibody against the human epidermal growth factor receptor 2 (HER2) receptor and was approved by the FDA in 1998 as a therapy for HER2-positive breast cancer patients (33, 34). The combination of doxorubicin with tratuzumab increases treatment efficacy but also is often accompanied by increased cardiotoxicity (35). The effects of BAI-1 were tested using the chronic doxorubicin model followed by the initiation one week later of trastuzumab (FIG. 26). Echocardiography was performed at week 8 following initiation of doxorubicin. Results show that doxorubicin+trastuzumab induced cardiac dysfunction was inhibited by BAI-1 as assessed using ejection fraction, fractional shortening and stroke volume (FIG. 27).

### BAI-1 Does Not Inhibit Doxorubicin-Induced Cancer Cell Death

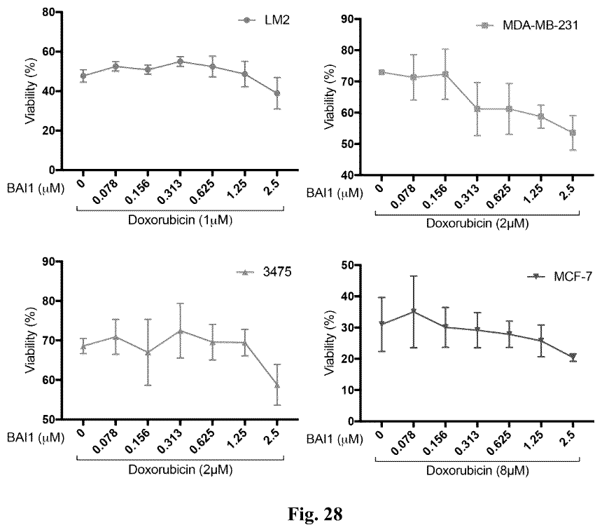

FIG. 28 illustrates human breast cancer cell lines LM2, MDA-MB-231, 3475 and MCF-7 treated with doxorubicin without or with varying concentrations of BAI-1. BAI-1 did not interfere with doxorubicin-induced killing of any of the breast cancer cells. Co-treatment with BAI-1 also did not compromise the cytotoxic effect of doxorubicin in an in vivo breast cancer xenograft mouse model (data not shown).

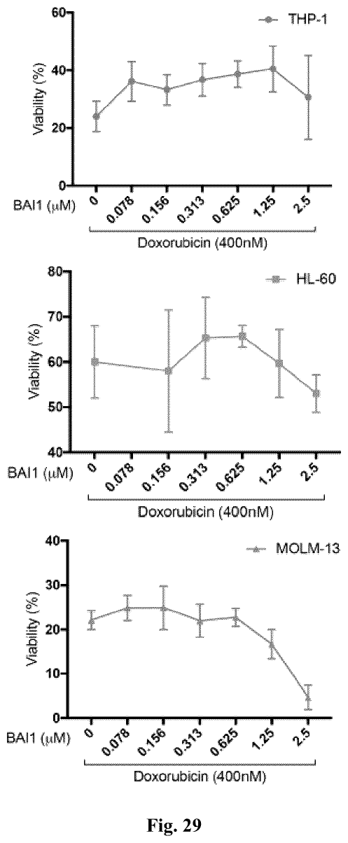

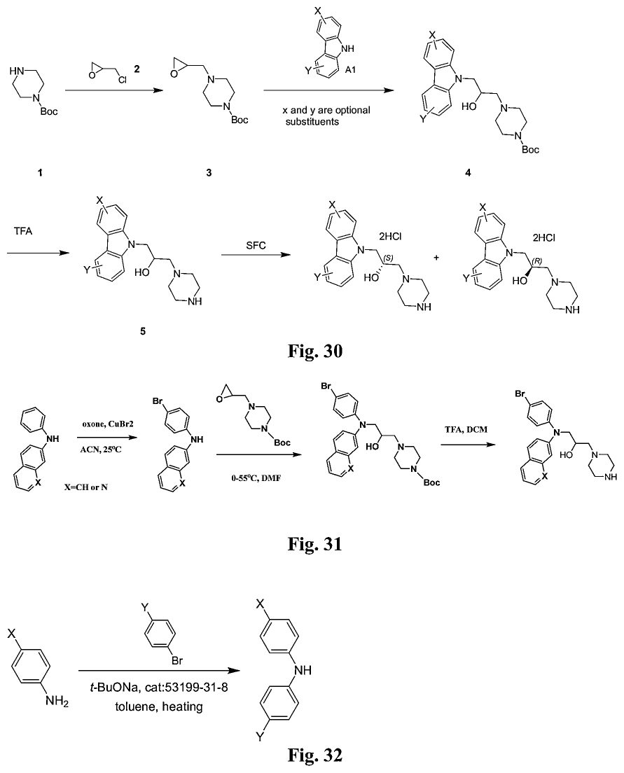

BAI-1 also did not effect of the ability of doxorubicin to kill acute myeloid leukemia (AML) cell lines in culture (FIG. 29). Co-treatment with BAI-1 also did not compromise the cytotoxic effect of doxorubicin in an in vivo AML heterograft mouse model (data not shown). BAI-1 protected against doxorubicin-induced cardiomyopathy in the same mice whose leukemia burden was successfully reduced by doxorubicin. These results indicate that BAI-1 can protect against doxorubicin-induced cardiomyopathy without interfering with reduction of leukemia burden in the same animals.

BAX levels were assessed in the adult heart versus the 4 breast cancer and 3 AML cell lines that were studied above. BAX levels were uniformly increased in the cancer cell lines compared with the heart. Next, to test the functional significance of the high BAX levels in tumor cells as a mechanism to escape BAI-1 from inhibiting doxorubicin-induced killing, BAX levels were knocked down in THP-1 AML cells using siRNA. While 73% reduction in BAX levels did not affect basal killing by doxorubicin, it resulted in BAI-1 interfering with doxorubicin-induced apoptosis (data not shwon). These data suggest that high BAX levels in tumors compared to the heart may be one mechanism by which BAI-1 protects the heart against doxorubicin without interfering with its killing of tumor cells.

TABLE 1
BAX inhibition activity of BAI compounds in liposomal
assays upon tBID-induced BAX activation.

| Inhibitor | Predicted IC50/μM | Measured IC 50/μM |
|---|---|---|
| BAI1 | 4.0 | 4.0 |
| BAI2 | 4.8 | 5.0 |
| BAI-A1 | >20 | 40.0 |
| BAI-A2 | 9.4 | 9.0 |
| BAI-A4 | 8.6 | — |
| BAI-A5 | 9.8 | — |
| BAI-A6 | 12.8 | — |
| BAI-A7 | 10.5 | — |
| BAI-A8 | 14.0 | — |
| BAI-A9 | 9.7 | — |
| BAI-A10 | 10.8 | — |
| BAI-A11 | 10.4 | — |
| BAI-A12 | 8.8 | — |
| BAI-A13 | 13.8 | 13.0 |
| BAI-A14 | 15.3 | — |
| BAI-A15 | 11.7 | 6.0 |
| BAI-A16 | — | 18.0 |
| BAI-A17 | — | 27.0 |
| BAI-A18 | — | 15.0 |
| BAI-A19 | — | 26.0 |
| BAI-A20 | — | >>100 |
| BAI-A21 | — | 4.5 |
| BAI-A22 | — | 11 |

IC50 values were measured using liposome release experiments using a minimum of 4 inhibitor concentrations around the IC50. Normalized inhibition values are the percentage inhibition of each compound normalized to BAI1, averaged over 5 and 10 μM inhibitor concentrations. IC50 were predicted based on of “Normalized inhibition values” correlated with measured IC50 values.

TABLE 2
BAI-A22 pharmacokinetic parameter estimates from NCA

| Parameter | Units | Estimate |
|---|---|---|
| t_{1/2} | hr | 5.04 |
| tₘₐₓ | hr | 0 |
| Cₘₐₓ | ng/mL | 318 |
| C₀ | ng/mL | 318 |
| MRTₗₐₛₜ | hr | 2.15 |
| CL | L/hr/kg | 1.91 |
| Vₛₛ | L/kg | 10.5 |
| AUCₗₐₛₜ ± SE | hr * ng/mL | 390 ± 42.7 |
| AUC_{∞} | hr * ng/mL | 524 |

Pharmacokinetic parameters from non-compartmental analysis using WinNonLin software.
C₀: maximum plasma concentration extrapolated to t = 0;
tₘₐₓ: time of maximum plasma concentration;
t_{1/2}: half-life;
MRTₗₐₛₜ: mean residence time, calculated to the last observable time point;
CL: clearance;
Vₛₛ: steady state volume of distribution;
AUCₗₐₛₜ: area under the curve, calculated to the last observable time point;
AUC_{∞}: area under the curve, extrapolated to infinity.

### Materials and Methods for the Chemical Syntheses

All chemical reagents and solvents were obtained from commercial sources (Aldrich, Acros, Fisher) and used without further purification unless otherwise noted. Anhydrous solvents (tetrahydrofurane, toluene, dichloromethane, diethyl ether) were obtained using a Pure Solv™ AL-258 solvent purification system. N,N-Dimethylformamide was degassed and dried over freshly activated 4 Å molecular sieves. Chromatography was performed on a Teledyne ISCO CombiFlash Rf 200i using disposable silica cartridges (4, 12, and 24 g). Analytical thin layer chromatography (TLC) was performed on aluminum-backed Silicycle silica gel plates (250 μm film thickness, indicator F254). Compounds were visualized using a dual wave length (254 and 365 nm) UV lamp, and/or staining with CAM (cerium ammonium molybdate) or KMnO₄ stains. NMR spectra were recorded on Bruker DRX 300 and DRX 600 spectrometers. ¹H and ¹³C chemical shifts (δ) are reported relative to tetramethyl silane (TMS, 0.00/0.00 ppm) as internal standard or to residual solvent (CD3OD: 3.31/49.00 ppm; CDCl₃: 7.26/77.16 ppm; dmso-d₆: 2.50/39.52 ppm; acetone-d₆: 2.05/29.84 ppm; acetonitrile-d₃: 1.94/1.32 ppm). Mass spectra were recorded on a Shimadzu LCMS 2010EV (direct injection unless otherwise noted).

3,6-difluoro-9H-carbazole (22), 3,6-bis (trifluoromethyl)-9H-carbazole (23), 3,6-dimethyl-9H-carbazole (24), and tert-butyl piperazine-1-carboxylate (25) were synthesized according to literature procedures. As an alternative to Pd-catalyzed aminations, requisite diaryl amnines can be conveniently prepared using Knochel's procedure (26).

### Typical Synthetic Procedure—Synthesis of BAI-A22

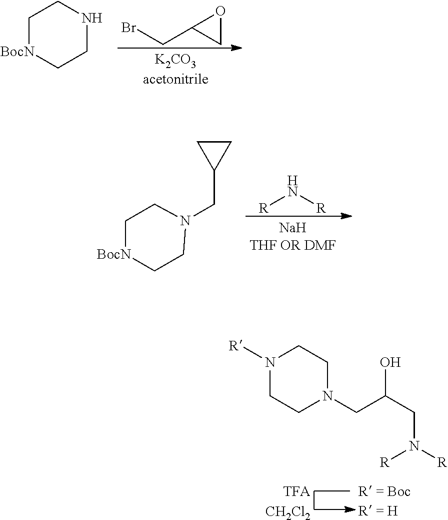

Scheme 1: Synthesis of BAI-A22 and its analogs. tert-Butyl piperazine-1-carboxylate was added to 2-(bromomethyl) oxirane to give intermediate. Amines were deprotonated using sodium hydride and used to open the epoxide. The resulting secondary alcohol was subjected to standard Boc deprotection conditions to obtain BAI-A22 and its analogs. (THF=tetrahydrofurane; DMF=N,N-dimethylformamide, TFA=trifluoroacetic acid; Boc=tert-butylcarboxylate)

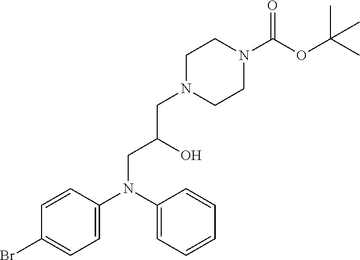 [SMILES: CC(C)(C)OC(=O)N1CCN(CC(O)CN(c2ccccc2)c2ccc(Br)cc2)CC1]

Synthesis of tert-butyl 4-(3-((4-bromophenyl) (phenyl) amino)-2-hydroxypropyl) piperazine-1-carboxylate. In a flame-dried 60 mL Centrifuge tube with septum and stir bar, sodium hydride (60% dispersion in mineral oil) (258 mg, 6.45 mmol, 1.60 equiv) was suspended in dry DMF (5.7 mL) under an argon atmosphere. In a separate dry and argon-flushed tube, 4-bromo-N-phenylaniline (1.50 g, 6.05 mmol, 1.50 equiv) was dissolved in dry DMF (18.4 mL). The NaH-suspension was cooled to 0° C. (ice bath) and the diphenylamine solution was slowly added over ca 10-15 min. A color change to bright yellow, later green was observed. After 20 min, the mixture was warmed to room temperatue (RT) and stirred for an additional 30 min. The mixture then was cooled to 0° C. again.

A solution of tert-butyl 4-(oxiran-2-ylmethyl) piperazine-1-carboxylate (0.977 g, 4.03 mmol) in dry DMF (2.83 mL) was added over 5 min. The mixture was stirred at 0° C. for 10 min, then warmed to RT and stirred at this temperature. Thin layer chromatography (TLC; 1:1 hex: EtOAc) was used to monitor the reaction progress. After TLC indicated full conversion, the mixture was poured onto satd. aq. sodium bicarbonate (75.0 mL), extracted with EtOAc (150 mL and 2×75.0 mL). Combined organic layers were dried (MgSO₄), filtered and evaporated in vacuo. The crude residue was purified on an Isco CombiFlash (silica gel, EtOAc in hexanes, 30%→>60%) (BAI-A22; 605 mg, 1.23 mmol, 31%) was obtained as off-white solid. The corresponding O-acetate (1.01 g) was isolated as a side-product. The acetate had presumably formed on the loading column from the desired product and ethyl acetate, triggered by heat formed when DMF remainders in the crude material came in contact with the silica. This behavior was not observed on a significant level in smaller scale reactions and can be avoided by more thorough drying of the crude in high vacuum (10⁻³ mbar). The acetate can be conveniently hydrolyzed by treatment with potassium carbonate (2.0 equiv) in methanol (0.11 M) for 2 h to give another crop of the desired product (685 mg, 35%).

TLC: R_{f} 0.37 (1:1, hex:EtOAc). ¹H-NMR (600 MHz, CDCl₃): δ 7.32-7.28 (m, 4H), 7.08 (d, J=7.9 Hz, 2H), 7.03 (t, J=7.4 Hz, 1H), 6.88 (d, J=8.9 Hz, 2H), 4.00 (dd, J=10.3, 5.7 Hz, 1H), 3.79-3.71 (m, 2H), 3.44-3.38 (m, 4H), 3.31 (s, 1H), 2.53-2.52 (m, 2H), 2.41 (dd, J=12.4, 3.5 Hz, 1H), 2.34 (m, 3H), 1.45 (s, 9H). ¹³C-NMR (151 MHz, CDCl₃): δ154.8, 147.9, 147.8, 132.2, 129.7, 122.9, 122.7, 121.6, 113.2, 80.0, 65.1, 62.27, 56.8, 53.2, 43.8 (d, br), 28.6. ESI-MS m/z (rel int): (pos) 514.1 ([M(⁸¹Br)+Na]⁺, 18), 512.1 ([M(⁷⁹Br)+Na]⁺, 14), 492.1 ([M(⁸¹Br)+H]⁺, 100), 490.1 ([M(⁷⁹Br)+H]⁺, 95), 436.0 (14), 435.0 (18).

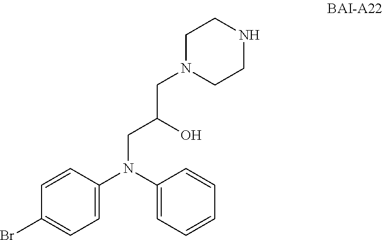 [SMILES: OC(CN1CCNCC1)CN(c1ccccc1)c1ccc(Br)cc1]

Synthesis of 1-((4-bromophenyl)(phenyl)amino)-3-(piperazin-1-yl)propan-2-ol (BAI-A22). tert-Butyl 4-(3-((4-bromophenyl)(phenyl)amino)-2-hydroxypropyl)piperazine-1-carboxylate (605 mg, 1.23 mmol) was dissolved in dichloromethane (20.6 mL). The flask was purged with argon for a few minutes, and TFA (5.23 mL, 67.8 mmol, 55.0 equiv) was added at RT and the mixture stirred at the same temperature. TLC analysis of a reaction aliquot (micro-workup, satd. aq. NaHCO3/EtOAc) indicated complete conversion after 1 h 05′. The mixture was poured on sodium bicarbonate (6.22 g, 74.0 mmol, 60.0 equiv) in 20.0 mL water and stirred vigorously at RT for 40 min. More sodium bicarbonate was added as needed to bring the aqueous layer to pH=8. The layers were separated and the aqueous layer was extracted with EtOAc (2×75.0 mL). The combined organic layers were washed with satd. aq. NaHCO₃ (50.0 mL) and brine (50.0 mL), dried (MgSO4), filtered and evaporated in vacuo. The residue was taken up in CH₂Cl₂, filtered through syringe filter (pore size), then evaporated in vacuo and dried in high vacuum (foams heavily!). Ethyl-3-oxo-3-phenyl-2-(2-(thiazol-2-yl) hydrazono) propanoate (470 mg, 1.20 mmol, 98%) was obtained as a sticky, light brown solid.

TLC: R_{f} 0.09 (95:5, CH₂Cl₂:MeOH). ¹H-NMR (600 MHZ, CDCl₃): δ 7.33-7.28 (m, 4H), 7.09 (dd, J=8.6, 1.1 Hz, 2H), 7.03 (tt, J=7.3, 1.1 Hz, 1H), 6.89 (d, J=9.0 Hz, 2H), 4.03-3.98 (m, 1H), 3.78-3.70 (m, J=5.7 Hz, 2H), 2.94-2.87 (m, 4H), 2.63-2.60 (m, 2H), 2.44-2.38 (m, 3H), 2.30 (dd, J=12.4, 10.1 Hz, 1H). ¹³C-NMR (151 MHz, CDCl₃): δ147.9, 147.8, 132.2, 129.7, 122.9, 122.7, 121.6, 113.2, 64.8, 62.7, 56.9, 54.2, 46.0. ESI-MS m/z (rel int): (pos) 392.09 ([M(⁸¹Br)+H]⁺, 100), 389.9 ([M(⁷⁹Br)+H]⁺, 99).

Final products can be converted into their (e.g., HCl) salts.

### Synthesis of Additional Compounds

The following compounds were prepared in an analogous fashion.

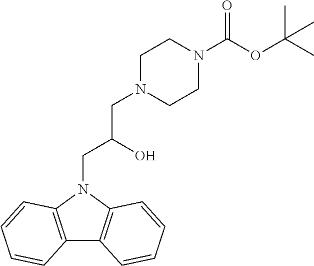 [SMILES: CC(C)(C)OC(=O)N1CCN(CC(O)Cn2c3ccccc3c3ccccc32)CC1]

TLC: R_{f} 0.20 (1:1, hex:EtOAc). ¹H-NMR (600 MHZ, CDCl₃): δ 8.10 (dt, J=7.7, 0.9 Hz, 2H), 7.50-7.46 (m, 4H), 7.26 (ddd, J=7.7, 6.0, 1.8 Hz, 3H), 4.40 (d, J=5.4 Hz, 2H), 4.28 (m, J=5.3 Hz, 1H), 3.45-3.36 (m, J=3.3 Hz, 4H), 2.54-2.45 (m, 4H), 2.32 (s, 2H), 1.44 (s, 9H). ¹³C-NMR (151 MHZ, CDCl₃): δ 154.8, 141.1, 125.9, 123.2, 120.4, 119.3, 109.2, 80.0, 66.5, 62.0, 53.1, 47.2, 43.7 (d, br), 28.5. ESI-MS m/z (rel int): (pos) 432.1 ([M+Na]⁺, 100); (neg) 444.2 ([M+Cl]⁻, 100).

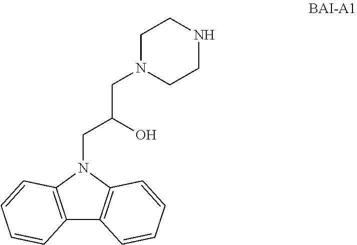 [SMILES: OC(CN1CCNCC1)Cn1c2ccccc2c2ccccc21]

TLC: R_{f} 0.24 (95:5, CH₂Cl₂:MeOH). ¹H-NMR (600 MHz, dmso-d₆): δ 8.13 (d, J=7.7 Hz, 2H), 7.63 (d, J=8.2 Hz, 2H), 7.43 (t, J=7.7 Hz, 2H), 7.18 (t, J=7.4 Hz, 2H), 4.99-4.91 (m, 1H), 4.48-4.45 (m, 1H), 4.29 (dd, J=14.8, 6.9 Hz, 1H), 4.10-4.03 (m, 1H), 2.86-2.80 (m, 4H), 2.44-2.32 (m, 6H). ¹³C-NMR (151 MHZ, dmso-d₆): δ 131.0, 115.7, 112.4, 110.3, 108.9, 100.2, 57.36, 52.5, 43.3, 37.9, 34.8. ESI-MS m/z (rel int): (pos) 310.0 ([M+H]⁺, 100).

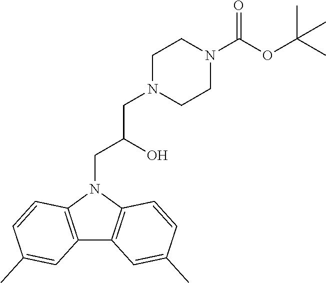 [SMILES: Cc1ccc2c(c1)c1cc(C)ccc1n2CC(O)CN1CCN(C(=O)OC(C)(C)C)CC1]

TLC: R_{f} 0.29 (1:1, hex:EtOAc). ¹H-NMR (600 MHZ, CDCl₃): δ 7.84 (s, 2H), 7.33 (d, J=7.1 Hz, 2H), 7.26 (d, J=7.1 Hz, 2H), 4.37-4.31 (m, 2H), 4.22-4.22 (br s, 1H), 3.39 (br s, 4H), 2.52-2.41 (m, 11H), 2.29 (br s, 2H). ¹³C-NMR (151 MHz, CDCl₃): δ 154.8, 139.7, 128.4, 127.0, 123.1, 120.4, 108.8, 80.0, 66.5, 62.0, 53.1, 47.2 (br d), 43.6, 28.5, 21.5. ESI-MS m/z (rel int): (pos) 460.2 ([M+Na]⁺, 34), 438.1 ([M+H]⁺, 100); (neg) 472.3 ([M+Cl]⁻, 45), 436.4 ([M−H]⁻, 10).

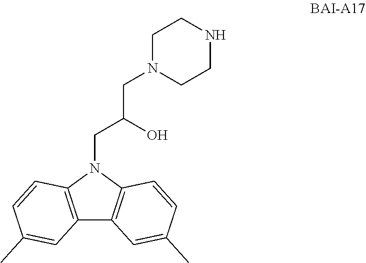 [SMILES: Cc1ccc2c(c1)c1cc(C)ccc1n2CC(O)CN1CCNCC1]

TLC: R_{f} 0.02 (95:5, CH₂Cl₂:MeOH). ¹H-NMR (600 MHZ, acetone-d₆): δ 7.86 (s, 2H), 7.49 (d, J=8.3 Hz, 2H), 7.23 (dd, J=8.3, 1.4 Hz, 2H), 4.48 (dd, J=14.8, 4.7 Hz, 1H), 4.35 (dd, J=14.8, 6.6 Hz, 1H), 4.30 (dddd, J=7.0, 6.6, 5.0, 4.7 Hz, 1H), 3.30-3.25 (m, 4H), 2.85-2.83 (m, 2H), 2.76 (dd, J=11.4, 6.2 Hz, 2H), 2.62 (dd, J=12.8, 5.0 Hz, 1H), 2.54 (dd, J=12.8, 7.0 Hz, 1H), 2.48 (s, 6H). ¹³C-NMR (151 MHZ, acetone-d₆): δ 140.6, 128.4, 127.5, 123.6, 120.6, 110.3, 68.27, 62.5, 51.5, 48.3, 44.37, 21.4. ESI-MS m/z (rel int): (pos) 338.00 ([M+Na]⁺, 100).

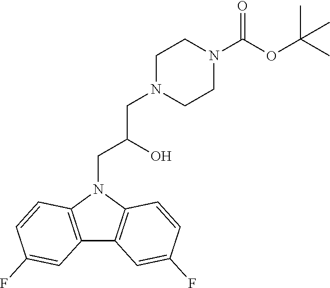 [SMILES: CC(C)(C)OC(=O)N1CCN(CC(O)Cn2c3ccc(F)cc3c3cc(F)ccc32)CC1]

TLC: R_{f} 0.29 (1:1, hex:EtOAc). ¹H-NMR (600 MHZ, CDCl₃): δ 7.66 (dd, J=8.7, 2.5 Hz, 2H), 7.39 (dd, J=8.9, 4.1 Hz, 2H), 7.20 (ddd, J=8.9, 8.7, 2.5 Hz, 2H), 4.35 (dd, J=15.3, 4.5 Hz, 1H), 4.29 (dd, J=15.3, 5.7 Hz, 1H), 4.18 (ddt, J=9.1, 5.6, 4.6 Hz, 1H), 3.46-3.35 (m, 5H), 2.53 (s, 2H), 2.45-2.39 (m, 2H), 2.30 (s, 2H), 1.44 (s, 9H). ¹³C-NMR (151 MHz, CDCl₃): δ 157.4 (d, J=236.4 Hz), 154.8, 138.4, 123.0 (dd, J=9.5, 4.1 Hz), 114.3 (d, J=25.4 Hz), 110.2 (d, J=8.9 Hz), 106.2 (d, J=23.4 Hz), 80.0, 66.6, 61.7, 53.1, 47.5, 43.7 (d, br), 28.5. ESI-MS m/z (rel int): (pos) 446.0 ([M+H]⁺, 100); (neg) 480.1 ([M+Cl]⁻, 100).

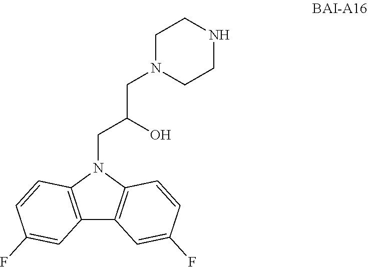 [SMILES: OC(CN1CCNCC1)Cn1c2ccc(F)cc2c2cc(F)ccc21]

TLC: R_{f} 0.02 (95:5, CH₂Cl₂:MeOH). ¹H-NMR (600 MHZ, acetone-d₆): δ 7.89 (dd, J=9.1, 2.6 Hz, 2H), 7.69 (dd, J=9.0, 4.3 Hz, 2H), 7.27 (ddd, J=9.1, 9.0, 2.6 Hz, 2H), 4.59 (dd, J=15.1, 4.0 Hz, 1H), 4.45 (dd, J=15.1, 7.0 Hz, 1H), 4.34-4.30 (m, 1H), 3.34-3.24 (m, 4H), 2.89-2.78 (m, 4H), 2.67 (dd, J=12.8, 5.5 Hz, 1H), 2.56 (dd, J=12.8, 6.9 Hz, 1H). ¹³C-NMR (151 MHz, acetone-d₆): δ 157.9 (d, J=233.3 Hz,), 139.5, 123.5 (dd, J=9.9, 4.3 Hz), 114.6 (d, J=25.4 Hz), 112.1 (d, J=8.9 Hz), 106.6 (d, J=24.1 Hz), 68.4, 62.4, 51.7, 48.6, 44.5. ESI-MS m/z (rel int): (pos) 346.0 ([M+H]⁺, 100).

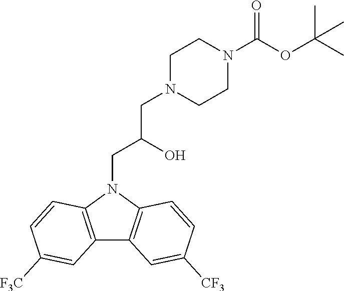 [SMILES: CC(C)(C)OC(=O)N1CCN(CC(O)Cn2c3ccc(C(F)(F)F)cc3c3cc(C(F)(F)F)ccc32)CC1]

TLC: R_{f} 0.33 (4:1, hex:EtOAc). ¹H-NMR (600 MHZ, CDCl₃): δ 8.39 (t, J=1.0 Hz, 2H), 7.75 (dd, J=8.6, 1.0 Hz, 2H), 7.61 (d, J=8.6 Hz, 2H), 4.47 (dd, J=15.3, 4.0 Hz, 1H), 4.38 (dd, J=15.3, 5.9 Hz, 1H), 4.21 (dddd, J=10.2, 6.0, 4.0, 3.8 Hz, 1H), 3.47 (br s, 1H), 3.50-3.36 (m, J=4.1 Hz, 4H), 2.58-2.53 (m, 2H), 2.49 (dd, J=12.3, 3.8 Hz, 1H), 2.41 (dd, J=12.3, 10.2 Hz, 1H), 2.34-2.30 (m, 2H), 1.44 (s, 9H). ¹³C-NMR (151 MHZ, CDCl₃): δ 154.8, 143.3, 125.1 (q, J=272.0 Hz), 123.6 (q, J=3.5 Hz), 122.6 (q, J=32.5Hz), 122.4, 118.3 (q, J=4.0 Hz), 110.1, 80.1, 66.5, 61.6, 53.1, 47.5, 43.7 (d, br), 28.5. ESI-MS m/z (rel int): (pos) 546.1 ([M+H]⁺, 100).

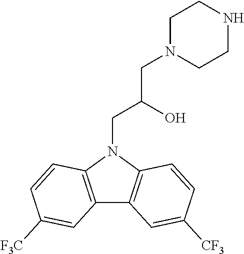 [SMILES: OC(CN1CCNCC1)Cn1c2ccc(C(F)(F)F)cc2c2cc(C(F)(F)F)ccc21]

TLC: R_{f} 0.00 (1:1, hex:EtOAc). ¹H-NMR (600 MHz, acetone-d₆): δ 8.74 (t, J=0.8 Hz, 2H), 7.98 (d, J=8.7 Hz, 2H), 7.84 (dd, J=8.7, 1.5 Hz, 2H), 4.76 (dd, J=15.1, 3.5 Hz, 1H), 4.61 (dd, J=15.1, 7.4 Hz, 1H), 4.38 (dddd, J=7.4, 6.8, 6.0, 3.5 Hz, 1H), 3.20 (t, J=5.1 Hz, 4H), 2.79-2.74 (m, 4H), 2.70 (dd, J=12.7, 6.0 Hz, 1H), 2.59 (dd, J=12.7, 6.8Hz, 1H). ¹³C-NMR (151 MHz, acetone-d₆): δ 143.7, 125.5 (q, J=270.6 Hz), 123.0 (q, J=3.5 Hz), 122.1, 121.3 (q, J=31.9 Hz), 118.3 (q, J=4.3 Hz), 111.2, 67.4, 61.7, 52.1, 47.9, 44.3. ESI-MS m/z (rel int): (pos) 446.0 ([M+H]⁺, 100).

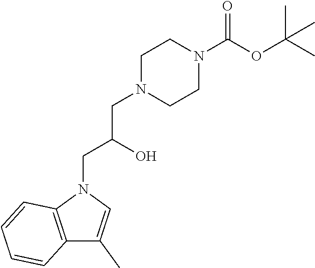 [SMILES: Cc1cn(CC(O)CN2CCN(C(=O)OC(C)(C)C)CC2)c2ccccc12]

TLC: R_{f} 0.30 (1:1, hex:EtOAc). ¹H-NMR (600 MHZ, CDCl₃): δ (d, J=7.8 Hz, 1H), 7.30 (d, J=8.2 Hz, 1H), 7.20 (t, J=7.6 Hz, 1H), 7.10 (t, J=7.4 Hz, 1H), 6.94 (s, 1H), 4.15 (dd, J=14.7, 4.7 Hz, 1H), 4.11 (dd, J=14.7, 5.5 Hz, 1H), 4.05 (dq, J=9.5, 4.8 Hz, 1H), 3.48-3.34 (m, 4H), 2.53-2.49 (m, 2H), 2.36-2.30 (m, 7H), 1.44 (s, 9H). ¹³C-NMR (151 MHz, CDCl₃): δ 154.8, 137.0, 128. 9, 126.5, 121.7, 119.2, 118.8, 110.9, 109.3, 80.0, 66.6, 61.5, 53.1, 49.7, 43.8 (d, br), 28.5, 9.8. ESI-MS m/z (rel int): (pos) 396.1 ([M+Na]⁺, 63), 374.0 ([M+H]⁺, 100).

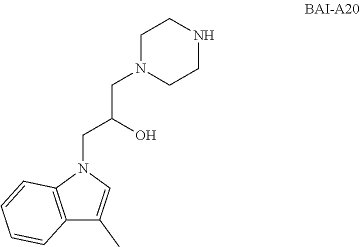 [SMILES: Cc1cn(CC(O)CN2CCNCC2)c2ccccc12]

TLC: R_{f} 0.03 (95:5, CH₂Cl₂:MeOH). ¹H-NMR (600 MHz, acetone-d₆): δ 7.49 (dd, J=7.8, 0.8 Hz, 1H), 7.43 (d, J=8.2 Hz, 1H), 7.12 (ddd, J=8.2, 7.1, 1.1 Hz, 1H), 7.07 (s, 1H), 7.02 (ddd, J=7.8, 7.1, 0.8 Hz, 1H), 4.68-4.64 (m, 1H), 4.32 (dd, J=14.7, 4.4 Hz, 1H), 4.23 (dd, J=14.7, 7.0 Hz, 1H), 3.98-3.86 (m, 4H), 3.81 (t, J=9.7 Hz, 4H), 3.57 (d, J=12.6 Hz, 1H), 3.39 (dd, J=12.6, 10.3 Hz, 1H), 2.26 (s, 3H), 2.05 (s, 4H). ¹³C-NMR (151 MHz, acetone-d₆): δ 137.9, 129.8, 127.5, 122.1, 122.1, 119.4 (2 carbons, confirmed by HSQC), 110.8, 110.4, 66.4, 61.1, 50.5, 50.3, 41.8, 9.6. ESI-MS m/z (rel int): (pos) 274.1 ([M+H]⁺, 100).

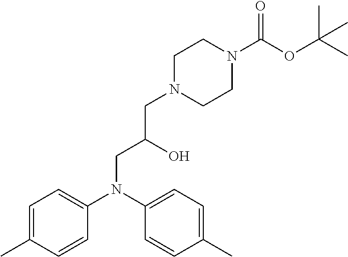 [SMILES: Cc1ccc(N(CC(O)CN2CCN(C(=O)OC(C)(C)C)CC2)c2ccc(C)cc2)cc1]

TLC: R_{f} 0.46 (1:1, hex:EtOAc). ¹H-NMR (600 MHz, CDCl₃): δ 7.05 (d, J=8.3 Hz, 4H), 6.91 (d, J=8.3 Hz, 4H), 4.01 (dtd, J=9.6, 5.9, 3.5 Hz, 1H), 3.73 (d, J=5.9 Hz, 2H), 3.43-3.36 (m, 4H), 2.54-2.48 (m, 2H), 2.45 (dd, J=12.5, 3.5 Hz, 1H), 2.37-2.30 (m, J=10.2 Hz, 3H), 2.29 (s, 6H), 1.45 (s, 9H). ¹³C-NMR (151 MHz, CDCl₃): δ 154.8, 146.4, 131.0, 130.0, 121.2, 79.9, 65.3, 62.4, 57.1, 53.3, 43.7 (d, br), 28.6, 20.8. ESI-MS m/z (rel int): (pos) 462.1 ([M+Na]⁺, 41), 440.1 ([M+H]⁺, 100).

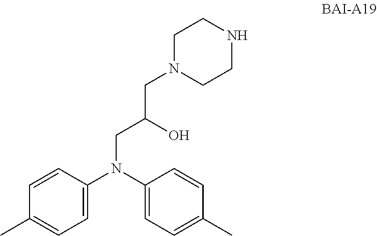

TLC: R_{f} 0.00 (1:1, hex:EtOAc). ¹H-NMR (600 MHz, acetone-d₆): δ 7.05 (d, J=8.3 Hz, 4H), 6.96 (d, J=8.3 Hz, 4H), 4.03 (dddd, J=7.5, 6.8, 4.9, 4.4 Hz, 1H), 3.91 (dd, J=14.9, 4.9 Hz, 1H), 3.62 (dd, J=14.9, 6.8 Hz, 1H), 3.20 (t, J=5.1 Hz, 4H), 2.83-2.79 (m, 2H), 2.73-2.70 (m, 2H), 2.58 (dd, J=12.8, 4.4 Hz, 1H), 2.48 (dd, J=12.8, 7.5 Hz, 1H), 2.25 (s, 6H). ¹³C-NMR (151 MHZ, acetone-d6): 8 147.4, 131.0, 130.4, 121.9, 66.9, 62.9, 57.7, 51.9, 44.6, 20.6. ESI-MS m/z (rel int): (pos) 340.0 ([M+H]⁺, 100).

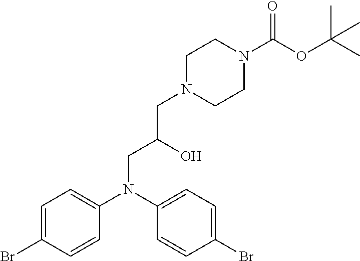 [SMILES: CC(C)(C)OC(=O)N1CCN(CC(O)CN(c2ccc(Br)cc2)c2ccc(Br)cc2)CC1]

TLC: R_{f} 0.60 (1:1, hex:EtOAc). ¹H-NMR (600 MHZ, CDCl₃): δ 7.35 (d, J=7.1 Hz, 4H) 6.94 (d, J=7.1 Hz, 4H), 3.99-3.95 (m, 1H), 3.75 (dd, J=15.3, 4.1 Hz, 1H), 3.66 (dd, J=15.3, 7.1 Hz, 1H), 3.44-3.38 (m, 4H), 2.54 (m, 2H), 2.39 (dd, J=12.4, 3.7 Hz, 1H), 2.36-2.29 (m, 3H), 1.45 (s, 9H). ¹³C-NMR (151 MHz, CDCl₃): δ 154.6, 147.0, 132.3, 122.9, 114.4, 79.8, 64.7, 62.0, 56.6, 53.1, 43.6 (d, br), 28.4. ESI-MS m/z (rel int): (pos) 568.0 ([M(⁸¹Br, ⁸¹Br)+H]⁺, 50) 570.0 ([M(⁸¹Br, ⁷⁹Br)+H]⁺, 100), 572.1 ([M(⁷⁹Br, ⁷⁹Br)+H]⁺, 50).

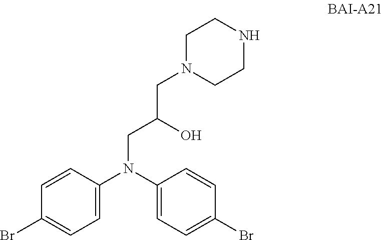 [SMILES: OC(CN1CCNCC1)CN(c1ccc(Br)cc1)c1ccc(Br)cc1]

TLC: R_{f} 0.00 (1:1, hex:EtOAc). ¹H-NMR (600 MHZ, CD₃CN): δ 7.38 (d, J=8.9 Hz, 4H), 7.00 (d, J=8.9 Hz, 4H), 3.94-3.90 (m, 1H), 3.86 (dd, J=15.3, 3.7 Hz, 1H), 3.57 (dd, J=15.3, 7.7 Hz, 1H), 3.14 (q, J=5.9 Hz, 4H), 2.75-2.73 ¹³C-NMR (151 MHZ, CD₃CN): δ 148.3, 133.0, 124.1, 114.3, 66.5, 62.1, 57.3, 51.1, 44.4, 1.32, 1.18. ESI-MS m/z (rel int): (pos) 471.9 ([M(⁸¹Br, ⁸¹Br)+H]⁺,50) 469.9 ([M(⁸¹Br, ⁷⁹Br)+H]⁺, 100), 467.9 ([M(⁷⁹Br, ⁷⁹Br)+H]⁺, 50).

What is claimed is:

1. A compound of formula A, or a pharmaceutically acceptable salt thereof, wherein the
compound is represented by Formula A 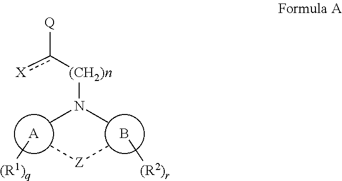
Wherein
A is optionally substituted phenyl, naphthyl, indene, or a 5-10-membered heteroaromatic ring having 1, 2 or 3 N atoms in the heteroaromatic ring;
B is optionally substituted phenyl, naphthyl, or a 5-10-membered heteroaromatic ring having 1, 2 or 3 N atoms in the heteroaromatic ring; or a 5-6-membered aliphatic ring with up to 3 heteroatoms;
R¹ and R² in each instance are independently selected from the group consisting of C1-C5 alkyl, F, Cl, Br, I, CN, NO₂, N(R⁴)₂, OR⁴, CF₃, COOH, COOR⁴, NHR⁴, OCR⁴, OCOR⁴, OR⁴, SR⁴, SOR⁴, and SO₂R⁴;
X is H, NH₂, OH, O, F, Cl, Br, I, CN, SH, NO₂, N(R⁴)₂, OR, CF₃, COOH, R⁴, COOR⁴, NHR⁴, OCR⁴, OCOR⁴, OR⁴, SR⁴, SOR⁴, or SO₂R⁴, wherein the bond between X and the main scaffold is a single bond or a double bond; alternatively, X is NR⁴ and the bond between X and the main scaffold is a double bond;
Q is 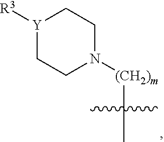
(CH₂)ₘN((CH₂)ₒR⁵)₂, COH, COOH, or CH₂NH(CH₂)₁OH;
R³ is none, H, C1-C6 alkyl, R4(C═O), or (CH₂)ₚOH;
R⁴ in each instance is independently H or C1-C3 alkyl;
R⁵ in each instance is independently OH, SH, NR4₂ or R4;
the dashed lines between A and Z and between B and Z indicates an optional bond;
Y is O, S, N or CH;
Z is void, O, S, NR4, CHR4, S(O)₂, C(Me)₂ or C(O), provided that when both of the dashed lines are absent, Z is void;
each l, m, n, o and p is independently 1, 2 or 3;
r is 0, 1, 2, 3 or 4; and
q is 0, 1, 2, 3 or 4.

2. The compound or the pharmaceutically acceptable salt thereof of claim 1, wherein Z is void and the dashed lines are absent.

3. The compound or the pharmaceutically acceptable salt thereof of claim 1, wherein R¹ and R² in each instance are independently selected from the group consisting of C1-C5 alkyl, F, Cl, Br and CF₃.

4. The compound or the pharmaceutically acceptable salt thereof of claim 1, wherein r is 0 or 1, q is 0 or 1.

5. The compound or the pharmaceutically acceptable salt thereof of claim 1, wherein X is OH, A is naphthyl, indene, or 10-membered heteroaromatic ring having 1, 2 or 3 N atoms in the heteroaromatic ring, wherein the naphthyl, indene, or 10-membered heteroaromatic ring is optionally substituted with one or more substituents selected from the group consisting of C1-C5 alkyl, F, Cl, Br, I, CN, N(R⁴)₂, OR⁴, CF₃, COOH, and COOR⁴.

6. The compound or the pharmaceutically acceptable salt thereof of claim 1, wherein A is optionally substituted naphthyl or indene.

7. The compound or the pharmaceutically acceptable salt thereof of claim 1, wherein A is

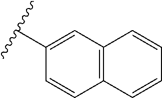 [SMILES: *c1ccc2ccccc2c1]

8. The compound or the pharmaceutically acceptable salt thereof of claim 5, wherein A is optionally substituted 10-membered heteroaromatic ring having 1 or 2 N atoms.

9. The compound or the pharmaceutically acceptable salt thereof of claim 5, wherein A is optionally substituted indole, quinoline or quinazoline.

10. The compound or the pharmaceutically acceptable salt thereof of claim 5, wherein A is

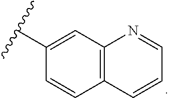 [SMILES: *c1ccc2cccnc2c1]

11. The compound or the pharmaceutically acceptable salt thereof of claim 5, wherein B is an optionally substituted phenyl.

12. The compound or the pharmaceutically acceptable salt thereof of claim 1, wherein A and B are each a phenyl substituted with an ethyl.

13. The compound or the pharmaceutically acceptable salt thereof of claim 1, wherein the compound is selected from the group consisting of

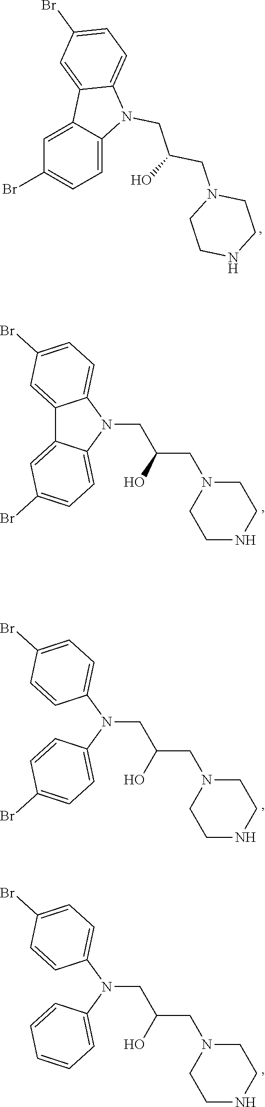 [SMILES: O[C@@H](CN1CCNCC1)Cn1c2ccc(Br)cc2c2cc(Br)ccc21 ; O[C@H](CN1CCNCC1)Cn1c2ccc(Br)cc2c2cc(Br)ccc21 ; OC(CN1CCNCC1)CN(c1ccc(Br)cc1)c1ccc(Br)cc1 ; OC(CN1CCNCC1)CN(c1ccccc1)c1ccc(Br)cc1]

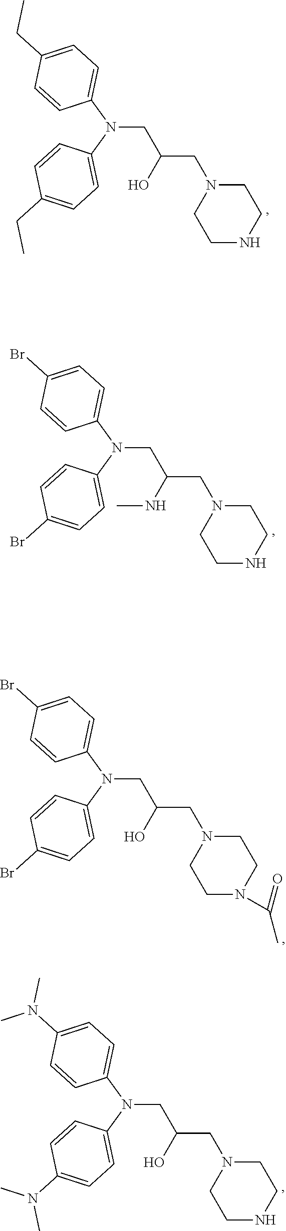 [SMILES: CCc1ccc(N(CC(O)CN2CCNCC2)c2ccc(CC)cc2)cc1 ; CNC(CN1CCNCC1)CN(c1ccc(Br)cc1)c1ccc(Br)cc1 ; CC(=O)N1CCN(CC(O)CN(c2ccc(Br)cc2)c2ccc(Br)cc2)CC1 ; CN(C)c1ccc(N(CC(O)CN2CCNCC2)c2ccc(N(C)C)cc2)cc1]

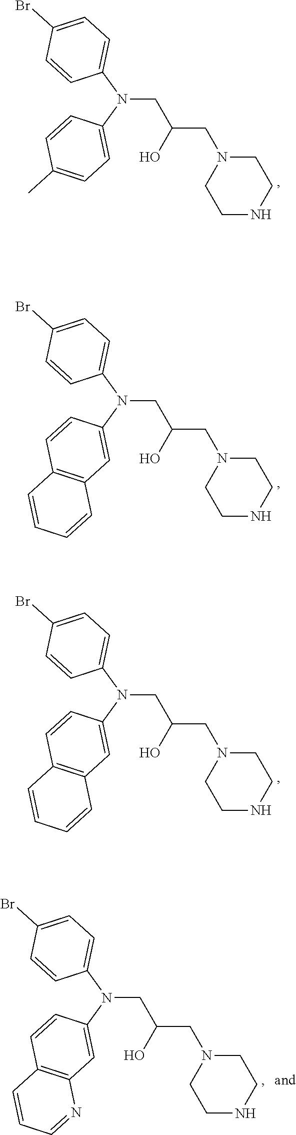 [SMILES: Cc1ccc(N(CC(O)CN2CCNCC2)c2ccc(Br)cc2)cc1 ; OC(CN1CCNCC1)CN(c1ccc(Br)cc1)c1ccc2ccccc2c1 ; OC(CN1CCNCC1)CN(c1ccc(Br)cc1)c1ccc2ccccc2c1 ; OC(CN1CCNCC1)CN(c1ccc(Br)cc1)c1ccc2cccnc2c1]

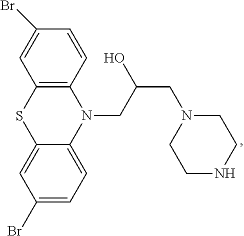 [SMILES: OC(CN1CCNCC1)CN1c2ccc(Br)cc2Sc2cc(Br)ccc21]

14. A pharmaceutical composition comprising a therapeutically effective amount of the compound or the pharmaceutically acceptable salt thereof of claim 1, and at least one pharmaceutically acceptable carrier.

15. A method of treating a disease associated with abnormal or deregulated mitochondrial outer-membrane permeabilization in a subject, comprising administering to the subject in need thereof a therapeutically effective amount of the compound or the pharmaceutically acceptable salt thereof of claim 1.

16. The method of claim 15, wherein the disease is selected from the group consisting of hypoxic cardiomyocytes, cardiac ischemia, cardiac ischemia-reperfusion injury, myocardial infarction, myocardial infarction and reperfusion injury, chemotherapy-induced cardiotoxicity, arteriosclerosis, heart failure, heart transplantation, aneurism, chronic pulmonary disease, ischemic heart disease, hypertension, pulmonary hypertension, thrombosis, cardiomyopathy, stroke, a neurodegenerative disease or disorder, an immunological disorder, ischemia, ischemia-reperfusion injury, infertility, a hematological disorder, renal hypoxia, hepatitis, a liver disease, a kidney disease, an intestinal disease, liver ischemia, intestinal ischemia, asthma, AIDS, Alzheimer's disease, Frontotemporal Dementia, Taupathies, Parkinson's disease, Huntington's disease, retinitis pigmentosa, spinal muscular atrophy, cerebellar degeneration, amyotrophic lateral sclerosis, organ transplant rejection, arthritis, lupus, irritable bowel disease, Crohn's disease, asthma, multiple sclerosis, diabetes, premature menopause, ovarian failure, follicular atresia, fanconi anemia, aplastic anemia, thalassemia, congenital neutropenia, myelodysplasia, a disease or disorder involving cell death and/or tissue damage.

17. The method of claim 16, wherein the disease or condition is chemotherapy-induced cardiotoxicity, and wherein the compound does not interfere with the ability of the chemotherapeutic agent to treat cancer.

18. The method of claim 17, wherein the chemotherapeutic agent is one or more of doxorubicin and trastuzumab.

19. The method of claim 14, wherein the disease is musculoskeletal disease or neurodegenerative diseases or retinal diseases or fibrosis associated with inflammation.

20. The method of claim 19, wherein the inflammation is associated with osteoarthritis, osteoporosis, rheumatoid arthritis, lupus, gout, neurodegenerative diseases (Alzheimer's disease, Frontotemporal Dementia, Taupathies, Parkinson's disease, Huntington's disease, amyotrophic lateral sclerosis,) and neuroinflammatory disorders.

21. A method of storing a biological material ex vivo or prolonging the viability of the biological material ex vivo for a period of time, comprising contacting the biological sample during the period with an effective amount of the compound or the pharmaceutically acceptable salt thereof of claim 1.

22. The method of claim 21, wherein more than 60% of the biological material remain viable after 14 days.

23. The method of any one of claims 21, wherein the biological material is selected from the group consisting of heart, lung, liver, kidney, spleen, stomach, intestine, pancreas, eye, bone, bone marrow, cochlea, and testis.
